SEQUENCE LISTING (22 sequences)

SEQ ID NO: 1 - DNA, 40100 bp, Artificial Sequence
  Synthetic Ad-PEPCK.Ribo-TK(PRT)
ttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccgggtgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgagcctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggtacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgcggttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgaatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaactgaaattaatcatgcagctgggagagtccttaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcgaccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggttagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactacaacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccggccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcttggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgtccttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccttggattacatcaagatcctctagttataactagagtacccggggatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccttgacaaaagaacccacactgattatgacacgcatactcggagctatgctaaccagcgtagccccgatgtaagctttcaatagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttcacagcggcagcctaacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtaacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatgttaattaacatgcatggatccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcagccatgagattatcaaaaaggatcttcacctagatccttttcacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattttgttaaaatttttgttaaatcagctcattttttaaccaataggccgaaatcggcaacatcccttataaatcaaaagaatagaccgcgatagggttgagtgttgttccagtttggaacaagagtccactattaaagaacgtggactccaacgtcaaagggcgaaaaaccgtctatcagggcgatggcccactacgtgaaccatcacccaaatcaagttttttgcggtcgaggtgccgtaaagctctaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgcgcgcttaatgcgccgctacagggcgcgtccattcgccattcaggatcgaattaattcttaattaacatcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatactgcgatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagtggcgcgccgtcgacgagctctttggggagtcctaagagggcagctggcaatggacacctagcagtccctttgagacttatttcagatggagctgtagaaagatgccatggctcacagtgcctccctgggaagggggcagagggctgcccagtgaggcctcttgcgagcaggaaatcaccagagacaaggaaagaccagaccccaggatgacctcagttaggccttgcccgactgtcctcagagtcccattctctgtgtcctggttcttttagaagatcatggacctccaggtcatttcgtaaccggaatctgcctgcggggggttttgacaagctatggtatagtgtatgtgggggtactgacgaattggaagatcatggagaccccttctcctcctccatcattggtctgccacatccctcccaggcgactcacagcagagagaccttggatgtatgtagggtgctttaaaactccagctgagttacagtctctcctttctgttttcaccttaaccttccagggatgcaaacccacgacaggtttagcagcagagtggaggctggccatgaatctcagagaaagtgctcactggaaaggctggtttagcccaggcctgatgtggaggcactgagctggacgttctagcggggttgacacccaacagtttacatagggggaggccacccctcctgagcagtctcggtgacttgaagaggaagccgcttcttctgtaccaacacagaagctccagcgaacccccagaatgctggcagtgtgggtgctatgtaaaagtatttacatagctttgtagagtgagccaagcccagtctgtttgggatgactcttcacagtgcctcgaatctgtcacacgtcttagtaagcagagtcacagagtttctgtcacatcatcctcctgcctacagggaagtaggccatgtccctgccccctactctgagcccagctgtgggagccagccctgcccaatgggctctctctgattggcttctcactcacttctaaactccagtgagcaacttctctcggctcgttcaattggcgtgaaggtctgtgtcttgcagagaaggttcttcacaactgggataaaggtctcgctgctcaagtgtagcccagtagaactgccaagccccttcccctcctctccctagactcttggatgcaagaagaatccaggcagctccaagggtgattgtgtccaacctagaatgtcttgaaaaagacattaaggggactagagaagacaggggatccaacggttctctgcagcccagcctgactgacatgtaactcttctggttctcaccagccagctggacctgcttagtattctttctgcctcagtttcccagcctgtacccagggctgtcatagttccatttcaggcagtagtaatgaatgagctgacataaaacatttagagcaggggtcagtatgtatatagagtgattattctatatcaggcattgcctcctcggaatgaagcttacaatcacccctccctctgcagttcatcttggggtggccagaggatccagcagacacctagtggggtaacacaccccagccaactcggctgttgcagactttgtctagaagtttcacgtctcagagctgaattcccttctcatgacctttggccgtgggagtgacacctcacagctgtggtgttttgacaaccagcagccactggcacacaaaatgtgcagccagcagcatatgaagtccaagaggcgtcccggccagccctgtccttgacccccacctgacaattaaggcaagagcctatagtttgcatcagcaacagtcacggtcaaagtttagtcaatcaaacgttgtgtaaggactcaactatggctgacacgggggcctgaggcctcccaacattcattaacaacagcaagttcaatcattatctccccaaagtttattgtgttaggtcagttccaaaccgtgctgaccatggctatgatccaaaggccggccccttacgtcagaggcgagcctccaggtccagctgaggggcagggctgtcctcccttctgtatactatttaaagcgaggagggctagctaccaagcacggttggccttccctctgggaacacacccttggccaacaggggaaatccggcgagacgctctgagatcctgcgagaaggaggtgcgtcctgctgcctgccccggcactctggctccccagctcaaggttcaggccttgccccaggccgggcctctgggtacctgaggtcttctcccgctctgtgcccttctcctcacctggctgcaatgagtgggggagcacggggcttctgcatgctgaaggcaccccactcagccaggcccttcttctcctccaggtcccccacggcccttcagatctgcggccgcaaggccagcacgttcttcgcgccgcgctcgcacagcctctgcagcactcgggccaccagctccttcaggcaggacacctggcggaaggagggggcggcggggggcggccgtgcgtcccagggcacgcacaccaggcactgggccaccagcgcgcggaaagccgccgggtccccgcgctgcaccagccgccagccctggggccccaggcgccgcacgaacgtggccagcggcagcacctcgcggtagtggctgcgcagcagggagcgcacggctaggcagcggggagcgcgcggcatcgcgggggtggccggggccagggcttcccaagcttcgttttgcggcaggaaaagttatcaggcatgcacctggtagctagtctttaaaccaatagattgcatcggtttaaaaggcaagaccgtcaaattgcgggaaaggggtcaacagccgttcagtaccaagtctcaggggaaactttgagatggccttgcaaagggtatggtaataagctgacggacatggtcctaaccacgcagccaagtcctaagtcaacagatcttctgttgatatggatgcagttcacagactaaatgtcggtcggggaagatgtattcttctcataagatatagtcggacctctccttaatgggagctagcggatgaagtgatgcaacactggagccgctgggaactaatttgtatgcgaaagtatattgattagttttggagtactcgcgaaaacgcccaccatggcttcgtacccctgccatcaacacgcgtctgcgttcgaccaggctgcgcgttctcgcggccatagcaaccgacgtacggcgttgcgccctcgccggcagcaagaagccacggaagtccgcctggagcagaaaatgcccacgctactgcgggtttatatagacggtcctcacgggatggggaaaaccaccaccacgcaactgctggtggccctgggttcgcgcgacgatatcgtctacgtacccgagccgatgacttactggcaggtgctgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggtgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcccgggagatgggggaggctaactgaagcggccgcgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgcaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtccgggttcaaaaccacttgctgggtggggagtcgtcagtaagtggctatgccccgaccccgaagcctgtttccccatctgtacaatggaaatgataaagacgcccatctgatagggtttttgtggcaaataaacatttggtttttttgttttgttttgttttgttttttgagatggaggtttgctctgtcgcccaggctggagtgcagtgacacaatctcatctcaccacaaccttcccctgcctcagcctcccaagtagctgggattacaagcatgtgccaccacacctggctaattttctatttttagtagagacgggtttctccatgttggtcagcctcagcctcccaagtaactgggattacaggcctgtgccaccacacccggctaattttttctatttttgacagggacggggtttcaccatgttggtcaggctggtctagaactcctgacctcaaatgatccacccacctaggcctcccaaagtgcacagattacaggcgtgggccaccgcacctggccaaatttttaatttttttctagagatagggtcttactgtgttgcccaggctggtgtcaaactcctgggctcaagcagatcctcctgcctcagcttcccaaagtggtgggattataggtgtgagccactgcgcccagtcagtagccccctctttgcccctcactgagccctactggatgttcttggttgtgtgacagtttccccatctattaaacagaaacccctatagcagaggggaggatgaggttggaaaatcaggagcattgttattctattcttgtgggatcggggaagcagacatctgggtggatgtttggggaatgctgggctcagttgaggaagtaggggggcccctggggcttacagggactggaagctctgagctggccagagggatgttgcaatcctgccagggtcttgtctatgctgtccctttcacaaccatccccctaccgccaggctgacacgtggttgtgggggcacaaggccagccgaactagagtctgaggctgggctgaggacaccctccccatcagctgccagggtcactggcggtcaaaggcagctggtggggaaggaattggactccagccctgggggacggatgtggtgatggtgggaagcaggcttggtgccaggaggggcatcagagggtgaataagagcagatagagtgtttgggggaggtagccagccaaagggggtgaggcccggtggaagggaagaaggggcatacactcagagctttgcagctgaaggttttaattttttgagatggggtctcactctgtctcaccaggctggagtgcagtggcgcaatcacagctcactgcagacgcgtgaattcctcgagtctagaggagcatgcgcgtcggcgcgccttcgaaggcgaaagatcccaacgaaaagagagaccacatggtccttcttgagtttgtaacagctgctgggattacacatggcatggatgaactgtacaactgaggatcccccgacctcgacctctggctaataaaggaaatttattttcattgcaatagtgtgttggaattttttgtgtctctcactcggaaggacatatgggagggcaaatcatttggtcgagatccctcggagatcggatctgggcgtgg

SEQ ID NO: 2 - DNA, 42159 bp, Artificial Sequence
  Synthetic Ad-PEPCK.Ribo-LacZ(PRL)
ttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccgggtgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgagcctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggtacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgcggttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgaatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaactgaaattaatcatgcagctgggagagtccttaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcgaccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggttagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactacaacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccggccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcttggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgtccttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccttggattacatcaagatcctctagttataactagagtacccggggatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccttgacaaaagaacccacactgattatgacacgcatactcggagctatgctaaccagcgtagccccgatgtaagctttcaatagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttcacagcggcagcctaacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtaacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatgttaattaacatgcatggatccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcagccatgagattatcaaaaaggatcttcacctagatccttttcacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattttgttaaaatttttgttaaatcagctcattttttaaccaataggccgaaatcggcaacatcccttataaatcaaaagaatagaccgcgatagggttgagtgttgttccagtttggaacaagagtccactattaaagaacgtggactccaacgtcaaagggcgaaaaaccgtctatcagggcgatggcccactacgtgaaccatcacccaaatcaagttttttgcggtcgaggtgccgtaaagctctaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgcgcgcttaatgcgccgctacagggcgcgtccattcgccattcaggatcgaattaattcttaattaacatcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatactgcgatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagtggcgcgccgtcgacgagctctttggggagtcctaagagggcagctggcaatggacacctagcagtccctttgagacttatttcagatggagctgtagaaagatgccatggctcacagtgcctccctgggaagggggcagagggctgcccagtgaggcctcttgcgagcaggaaatcaccagagacaaggaaagaccagaccccaggatgacctcagttaggccttgcccgactgtcctcagagtcccattctctgtgtcctggttcttttagaagatcatggacctccaggtcatttcgtaaccggaatctgcctgcggggggttttgacaagctatggtatagtgtatgtgggggtactgacgaattggaagatcatggagaccccttctcctcctccatcattggtctgccacatccctcccaggcgactcacagcagagagaccttggatgtatgtagggtgctttaaaactccagctgagttacagtctctcctttctgttttcaccttaaccttccagggatgcaaacccacgacaggtttagcagcagagtggaggctggccatgaatctcagagaaagtgctcactggaaaggctggtttagcccaggcctgatgtggaggcactgagctggacgttctagcggggttgacacccaacagtttacatagggggaggccacccctcctgagcagtctcggtgacttgaagaggaagccgcttcttctgtaccaacacagaagctccagcgaacccccagaatgctggcagtgtgggtgctatgtaaaagtatttacatagctttgtagagtgagccaagcccagtctgtttgggatgactcttcacagtgcctcgaatctgtcacacgtcttagtaagcagagtcacagagtttctgtcacatcatcctcctgcctacagggaagtaggccatgtccctgccccctactctgagcccagctgtgggagccagccctgcccaatgggctctctctgattggcttctcactcacttctaaactccagtgagcaacttctctcggctcgttcaattggcgtgaaggtctgtgtcttgcagagaaggttcttcacaactgggataaaggtctcgctgctcaagtgtagcccagtagaactgccaagccccttcccctcctctccctagactcttggatgcaagaagaatccaggcagctccaagggtgattgtgtccaacctagaatgtcttgaaaaagacattaaggggactagagaagacaggggatccaacggttctctgcagcccagcctgactgacatgtaactcttctggttctcaccagccagctggacctgcttagtattctttctgcctcagtttcccagcctgtacccagggctgtcatagttccatttcaggcagtagtaatgaatgagctgacataaaacatttagagcaggggtcagtatgtatatagagtgattattctatatcaggcattgcctcctcggaatgaagcttacaatcacccctccctctgcagttcatcttggggtggccagaggatccagcagacacctagtggggtaacacaccccagccaactcggctgttgcagactttgtctagaagtttcacgtctcagagctgaattcccttctcatgacctttggccgtgggagtgacacctcacagctgtggtgttttgacaaccagcagccactggcacacaaaatgtgcagccagcagcatatgaagtccaagaggcgtcccggccagccctgtccttgacccccacctgacaattaaggcaagagcctatagtttgcatcagcaacagtcacggtcaaagtttagtcaatcaaacgttgtgtaaggactcaactatggctgacacgggggcctgaggcctcccaacattcattaacaacagcaagttcaatcattatctccccaaagtttattgtgttaggtcagttccaaaccgtgctgaccatggctatgatccaaaggccggccccttacgtcagaggcgagcctccaggtccagctgaggggcagggctgtcctcccttctgtatactatttaaagcgaggagggctagctaccaagcacggttggccttccctctgggaacacacccttggccaacaggggaaatccggcgagacgctctgagatcctgcgagaaggaggtgcgtcctgctgcctgccccggcactctggctccccagctcaaggttcaggccttgccccaggccgggcctctgggtacctgaggtcttctcccgctctgtgcccttctcctcacctggctgcaatgagtgggggagcacggggcttctgcatgctgaaggcaccccactcagccaggcccttcttctcctccaggtcccccacggcccttcagatctgcggccgcaaggccagcacgttcttcgcgccgcgctcgcacagcctctgcagcactcgggccaccagctccttcaggcaggacacctggcggaaggagggggcggcggggggcggccgtgcgtcccagggcacgcacaccaggcactgggccaccagcgcgcggaaagccgccgggtccccgcgctgcaccagccgccagccctggggccccaggcgccgcacgaacgtggccagcggcagcacctcgcggtagtggctgcgcagcagggagcgcacggctaggcagcggggagcgcgcggcatcgcgggggtggccggggccagggcttcccaagcttcgttttgcggcaggaaaagttatcaggcatgcacctggtagctagtctttaaaccaatagattgcatcggtttaaaaggcaagaccgtcaaattgcgggaaaggggtcaacagccgttcagtaccaagtctcaggggaaactttgagatggccttgcaaagggtatggtaataagctgacggacatggtcctaaccacgcagccaagtcctaagtcaacagatcttctgttgatatggatgcagttcacagactaaatgtcggtcggggaagatgtattcttctcataagatatagtcggacctctccttaatgggagctagcggatgaagtgatgcaacactggagccgctgggaactaatttgtatgcgaaagtatattgattagttttggagtactcgcgaaaacgcccaccatggcttcgtacccctgccatcaacacgcgtctgcgttcgaccaggctgcgcgttctcgcggccatagcaaccgacgtacggcgttgcgccctcgccggcagcaagaagccacggaagtccgcctggagcagaaaatggtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataactgaagcggccgcgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgcaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtccgggttcaaaaccacttgctgggtggggagtcgtcagtaagtggctatgccccgaccccgaagcctgtttccccatctgtacaatggaaatgataaagacgcccatctgatagggtttttgtggcaaataaacatttggtttttttgttttgttttgttttgttttttgagatggaggtttgctctgtcgcccaggctggagtgcagtgacacaatctcatctcaccacaaccttcccctgcctcagcctcccaagtagctgggattacaagcatgtgccaccacacctggctaattttctatttttagtagagacgggtttctccatgttggtcagcctcagcctcccaagtaactgggattacaggcctgtgccaccacacccggctaattttttctatttttgacagggacggggtttcaccatgttggtcaggctggtctagaactcctgacctcaaatgatccacccacctaggcctcccaaagtgcacagattacaggcgtgggccaccgcacctggccaaatttttaatttttttctagagatagggtcttactgtgttgcccaggctggtgtcaaactcctgggctcaagcagatcctcctgcctcagcttcccaaagtggtgggattataggtgtgagccactgcgcccagtcagtagccccctctttgcccctcactgagccctactggatgttcttggttgtgtgacagtttccccatctattaaacagaaacccctatagcagaggggaggatgaggttggaaaatcaggagcattgttattctattcttgtgggatcggggaagcagacatctgggtggatgtttggggaatgctgggctcagttgaggaagtaggggggcccctggggcttacagggactggaagctctgagctggccagagggatgttgcaatcctgccagggtcttgtctatgctgtccctttcacaaccatccccctaccgccaggctgacacgtggttgtgggggcacaaggccagccgaactagagtctgaggctgggctgaggacaccctccccatcagctgccagggtcactggcggtcaaaggcagctggtggggaaggaattggactccagccctgggggacggatgtggtgatggtgggaagcaggcttggtgccaggaggggcatcagagggtgaataagagcagatagagtgtttgggggaggtagccagccaaagggggtgaggcccggtggaagggaagaaggggcatacactcagagctttgcagctgaaggttttaattttttgagatggggtctcactctgtctcaccaggctggagtgcagtggcgcaatcacagctcactgcagacgcgtgaattcctcgagtctagaggagcatgcgcgtcggcgcgccttcgaaggcgaaagatcccaacgaaaagagagaccacatggtccttcttgagtttgtaacagctgctgggattacacatggcatggatgaactgtacaactgaggatcccccgacctcgacctctggctaataaaggaaatttattttcattgcaatagtgtgttggaattttttgtgtctctcactcggaaggacatatgggagggcaaatcatttggtcgagatccctcggagatcggatctgggcgtgg

SEQ ID NO: 3 - DNA, 225 bp, Artificial Sequence
  Synthetic PEPCK promoter
aagtttattgtgttaggtcagttccaaaccgtgctgaccatggctatgatccaaaggccggccccttacgtcagaggcgagcctccaggtccagctgaggggcagggctgtcctcccttctgtatactatttaaagcgaggagggctagctaccaagcacggttggccttccctctgggaacacacccttggccaacaggggaaatccggcgagacgctctgaga

SEQ ID NO: 4 - DNA, 415 bp, Artificial Sequence
  Synthetic PEPCK enhancer
tcgaatctgtcacacgtcttagtaagcagagtcacagagtttctgtcacatcatcctcctgcctacagggaagtaggccatgtccctgccccctactctgagcccagctgtgggagccagccctgcccaatgggctctctctgattggcttctcactcacttctaaactccagtgagcaacttctctcggctcgttcaattggcgtgaaggtctgtgtcttgcagagaaggttcttcacaactgggataaaggtctcgctgctcaagtgtagcccagtagaactgccaagccccttcccctcctctccctagactcttggatgcaagaagaatccaggcagctccaagggtgattgtgtccaacctagaatgtcttgaaaaagacattaaggggactagagaagacaggggatcc

SEQ ID NO: 5 - DNA, 732 bp, Artificial Sequence
  Synthetic Rib21AS
aaggccagcacgttcttcgcgccgcgctcgcacagcctctgcagcactcgggccaccagctccttcaggcaggacacctggcggaaggagggggcggcggggggcggccgtgcgtcccagggcacgcacaccaggcactgggccaccagcgcgcggaaagccgccgggtccccgcgctgcaccagccgccagccctggggccccaggcgccgcacgaacgtggccagcggcagcacctcgcggtagtggctgcgcagcagggagcgcacggctaggcagcggggagcgcgcggcatcgcgggggtggccggggccagggcttcccaagcttcgttttgcggcaggaaaagttatcaggcatgcacctggtagctagtctttaaaccaatagattgcatcggtttaaaaggcaagaccgtcaaattgcgggaaaggggtcaacagccgttcagtaccaagtctcaggggaaactttgagatggccttgcaaagggtatggtaataagctgacggacatggtcctaaccacgcagccaagtcctaagtcaacagatcttctgttgatatggatgcagttcacagactaaatgtcggtcggggaagatgtattcttctcataagatatagtcggacctctccttaatgggagctagcggatgaagtgatgcaacactggagccgctgggaactaatttgtatgcgaaagtatattgattagttttggagtactcg

SEQ ID NO: 6 - DNA, 1127 bp, Artificial Sequence
  Synthetic HSV-tk
atggcttcgtacccctgccatcaacacgcgtctgcgttcgaccaggctgcgcgttctcgcggccatagcaaccgacgtacggcgttgcgccctcgccggcagcaagaagccacggaagtccgcctggagcagaaaatgcccacgctactgcgggtttatatagacggtcctcacgggatggggaaaaccaccaccacgcaactgctggtggccctgggttcgcgcgacgatatcgtctacgtacccgagccgatgacttactggcaggtgctgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggtgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcccgggagatgggggaggctaa

SEQ ID NO: 7 - DNA, 3051 bp, Artificial Sequence
  Synthetic LacZ
atggtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataa

SEQ ID NO: 8 - DNA, 20 bp, Artificial Sequence
  Synthetic HSV-tk forward primer
gcgaacatctacaccacaca

SEQ ID NO: 9 - DNA, 19 bp, Artificial Sequence
  Synthetic HSV-tk reverse primer
agttagcctcccccatctc

SEQ ID NO: 10 - DNA, 26 bp, Artificial Sequence
  Synthetic ITR forward primer
ggaattctggagtttgtgacgtggcg

SEQ ID NO: 11 - DNA, 32 bp, Artificial Sequence
  Synthetic ITR reverse primer
gctctagatggccaaatcttactcggttacgc

SEQ ID NO: 12 - DNA, 20 bp, Artificial Sequence
  Synthetic GAPDH forward primer
tgacatcaagaaggtggtga

SEQ ID NO: 13 - DNA, 20 bp, Artificial Sequence
  Synthetic GAPDH reverse primer
tccaccaccctgttgctgta

SEQ ID NO: 14 - DNA, 26 bp, Artificial Sequence
  Synthetic HSV-tk primer
cgggatcctcagttagcctcccccat

SEQ ID NO: 15 - DNA, 25 bp, Artificial Sequence
  Synthetic hTERT RNA forward primer
ggggaattcagcgctgcgtcctgct

SEQ ID NO: 16 - DNA, 20 bp, Artificial Sequence
  Synthetic HSV-tk reverse primer
gttatctgggcgcttgtcaa

SEQ ID NO: 17 - DNA, 18 bp, Artificial Sequence
  Synthetic 5′ primer specific for the trans-splicing junction
gctgcgtcctgctaaaac

SEQ ID NO: 18 - DNA, 20 bp, Artificial Sequence
  Synthetic nested 3′ primer specific to the HSV-tk
cagtagcgtgggcattttct

SEQ ID NO: 19 - DNA, 20 bp, Artificial Sequence
  Synthetic hTERT forward primer
cggaagagtgtctggagcaa

SEQ ID NO: 20 - DNA, 19 bp, Artificial Sequence
  Synthetic hTERT reverse primer
ggatgaagcggagtctgga

SEQ ID NO: 21 - DNA, 67 bp, Artificial Sequence
  Synthetic
gccgcgaattcactagtgattgctgcgtcctgctaaaacgcccaccatggcttcgtacccctgccat

SEQ ID NO: 22 - DNA, 32 bp, Artificial Sequence
  Synthetic
gctgcgtcctgctaaaacgcccaccatggctt

(19) United States
(12) Patent Application Publication
(10) Pub. No.: US 2011/0256524 A1
(43) Pub. Date: Oct. 20, 2011

(54) RECOMBINANT ADENOVIRUS COMPRISING TISSUE-SPECIFIC PROMOTER AND TUMOR-TARGETING TRANS-SPLICING RIBOZYME AND USES THEREOF

(75) Inventors: Seong-Wook Lee, Seoul (KR); Min-Sun Song, Seoul (KR); In-Hoo Kim, Goyang-si (KR); Jin-Sook Jeong, Busan (KR)

(73) Assignee: Industry-Academic Cooperation Foundation, Dankook University, Yongin-si (KR)

(21) Appl. No.: 12/599,671

(22) PCT Filed: Aug. 27, 2008
(86) PCT No.: PCT/KR08/05030

§ 371 Date: Nov. 19, 2009

(30) Foreign Application Priority Data
Aug. 25, 2008 (KR) 10-2008-0082732

(51) Int. Cl. C12Q 1/70; C12N 7/01

(52) U.S. Cl. 435/5; 435/235.1

(57) ABSTRACT

Disclosed herein are a recombinant adenovirus comprising tissue-specific promoters and trans-splicing ribozymes targeting tumor-specific genes, and uses thereof. More specifically, disclosed herein are a recombinant adenovirus comprising (1) a tissue-specific promoter, (2) a trans-splicing ribozyme acting on tumor-specific genes operably linked to the promoter, and (3) a therapeutic or reporter gene linked to 3′ exon of the ribozyme, an anticancer pharmaceutical composition comprising the same, and a composition for cancer diagnosis comprising the same.

The recombinant adenovirus exhibits high specificity and significantly improved therapeutic efficacy to gene targeted tissues. Accordingly, the recombinant adenovirus is useful as a gene delivery vector for anticancer agents or cancer diagnostics.

### TECHNICAL FIELD

The present invention relates to a recombinant adenovirus comprising tissue-specific promoters and trans-splicing ribozymes targeting tumor-specific genes, and uses thereof.

### BACKGROUND ART

Cancer is a serious disease, which is the leading cause of death in Korea and is an incurable disease, a comprehensive treatment for which has yet to be discovered in spite of a great deal of research. Conventional treatments associated with cancers include surgical operations, chemotherapy and radiotherapy. However, since these treatments have many limitations, other treatments having different concepts are studied at present, and in particular, gene therapy is actively researched.

Gene therapy refers to a method for genetically treating congenital or acquired gene abnormalities which are difficult to treat through conventional methods. Specifically, gene therapy is the insertion of genetic material such as DNA and RNA into the human body to express therapeutic proteins or inhibit expression of specific proteins, for treatment or prevention of chronic diseases such as congenital or acquired gene defects, viral diseases, cancers or cardiovascular diseases. Gene therapy fundamentally treats diseases by analyzing genetic causes thereof, thus being considered a promising method for treating incurable diseases and an alternative of conventional medical therapy.

Gene therapy for cancers is classified into immunogenic gene therapy wherein induce an immune response in the human body and direct gene therapy wherein the used genes directly kill cancer cells or induce death thereof. For the direct gene therapy, vectors to transmit genes into cells and express the same therein have a considerably important role. Adenovirus vectors are considered the most promising vectors for cancer gene therapy, because they exhibit high gene delivery efficiency, the ability to deliver genes into undifferentiated cells, and easy preparation of high-titer viral stocks.

Generally used adenovirus vectors for gene therapy delete a series of genes required for replication and insert highly promoter-active cytomegalovirus (CMV) or rous sarcoma virus (RSV) promoters to induce high expression of proteins to be treated.

Recently, in an attempt to reduce side effects occurred by expression of a great deal of target genes applicable to gene therapy in normal cells which frequently undergo cell division, cancer-tissue specific therapy was conducted (Fukuzawa et al., Cancer Res 64:363-369, 2004). For this purpose, methods using tissue-specific promoters, instead of CMV or RSV, are considered, but these methods have not yet been put to practical use due to the disadvantages of deteriorated therapeutic efficacy in spite of increased specificity. According to research associated with conventional tumor-specific gene promoters, AFP-, CEA-, PSA- or hTERT (human telomerase reverse transcriptase) promoters, these promoters were reported to exhibit an about 50 to 300 times decrease in gene expression capability, as compared to CMV-promoters (Kuhnnel et al., Cancer Gene Therapy 11:28-40, 2004).

In addition, an attempt to improve tissue specificity of gene therapy using tissue-specific promoters was already reported, but efficiency thereof is deteriorated and therapeutic efficacy thereof is thus decreased, as compared to general promoters (Wu, L., et al., Trends Mol. Med. 9:421-429, 2003).

Meanwhile, it was reported that two separate transcripts can be spliced to each other by trans-splicing Group I intron ribozymes from Tetrahymena thermophila both in vitro and in bacteria and human cells (Been, M. and Cech, T. 1986, One binding site determines sequence specificity of Tetrahymena pre-rRNA self-splicing, trans-splicing, and RNA enzyme activity. Cell 47: 207-216; Sullenger, B. A. and Cech, T. R. 1994, Ribozyme-mediated repair of defective mRNA by targeted, trans-splicing. Nature 371: 619-622; Jones, J. T., Lee, S. W., and Sullenger, B. A. 1996, Tagging ribozyme reaction sites to follow trans-splicing in mammalian cells. Nat Med. 2: 643-648). Accordingly, trans-splicing ribozymes based on Group I introns target specific RNAs, e.g., disease-associated gene transcripts or RNAs which are not expressed in normal cells but are specifically expressed only in disease cells, induce reprograms to modify the RNAs into normal RNAs or substitute the same by new therapeutic gene transcripts, thereby realizing highly disease-specific and safe gene therapy. That is, since RNA substitution occurs only when target gene transcripts are present, the desired resulting gene yields are obtained only under desired time and location conditions. In particular, the method involves substitution of the target RNA expressed in cells by the desired gene yields, thus controlling an expression level of genes to be introduced. In addition, trans-splicing ribozymes induce expression of desired therapeutic genes, while removing disease-specific RNA, thus improving therapeutic efficacy.

In addition, human telomerase reverse transcriptase (hTERT) is an important enzyme which regulates immortality and proliferation of tumor cells. This telomerase exhibits 80 to 90% telomerase activity to unlimitedly replicated germ cells, hematopoietic cells and tumor cells, but the normal cells around the tumor cells have no such activity (Bryan, T. M. and Cech, T. R. 1999, Telomerase and the maintenance of chromosome ends. Curr. Opin. Cell Biol. 11; 318-324). An attempt to inhibit proliferation of tumor cells by developing telomerase inhibitors that mediate cell growth through the telomerase property is actively underway (Bryan, T. M., Englezou, A., Gupta, J., Bacchetti, S., and Reddel, R. R. 1995, Telomere elongation in immortal human cells without detectable telomerase activity. Embo J. 14; 4240-4248; Artandi, S. E. and DePinho, R. A. 2000, Mice without telomerase: what can they teach us about human cancer Nat. Med. 6; 852-855).

The inventors of the present invention studied recombinant adenoviruses with improved therapeutic efficacy as well as tissue-specificity. As a result, a recombinant adenovirus was manufactured, which comprises a tissue-specific promoter; a trans-splicing ribozyme acting on tumor-specific genes operably linked to the promoter, and a therapeutic gene (or reporter gene) linked to the 3′ exon of the ribozyme. The present inventors found that the recombinant adenovirus cannot operate in other tissues except cancer-developed tissues, thus significantly reducing adverse effects caused by gene therapy and exhibiting high anti-cancer activity. The present invention has been completed based on the discovery.

### DISCLOSURE

### Technical Problem

Therefore, the present invention has been made in view of the above problems, and it is an object of the present invention to provide a recombinant adenovirus with improved tissue-specificity as well as therapeutic efficacy and uses thereof.

### Technical Solution

In accordance with one aspect of the present invention, the above and other objects can be accomplished by the provision of a recombinant adenovirus comprising: (1) a tissue-specific promoter; (2) a trans-splicing ribozyme acting on tumor-specific genes operably linked to the promoter; and (3) a therapeutic or reporter gene linked to the 3′ exon of the ribozyme.

In accordance with another aspect of the present invention, there is provided an anticancer pharmaceutical composition comprising the recombinant adenovirus as an active ingredient.

In accordance with another aspect of the present invention, there is provided a composition for cancer diagnosis comprising the recombinant adenovirus as an active ingredient.

In accordance with another aspect of the present invention, there is provided a cancer imaging method comprising: S1) introducing the recombinant adenoviruses into cancer cells; and S2) detecting reporter proteins from the cancer cells.

### Advantegeous Effect

The recombinant adenovirus of the present invention controls expression of trans-splicing ribozymes acting on tumor-specific genes due to the tissue-specific promoter and thus converts tumor-specific genes into therapeutic genes (or reporter genes) through trans-splicing reactions due to ribozyme expression in specific tissues, thereby selectively treating or diagnosing only cancer cells.

### DESCRIPTION OF DRAWINGS

The above and other objects, features and other advantages of the present invention will be more clearly understood from the following detailed description taken in conjunction with the accompanying drawings, in which:

FIG. 1 is a schematic diagram illustrating a recombinant adenovirus according to the present invention;

FIG. 2 is a gene map of the recombinant adenovirus manufactured in Examples of the present invention;

FIG. 3 is a graph illustrating MTS assay results ascertaining cell viability after Hep3B, SKOV3 and THLE3 cell lines were infected with Ad-PEPCK.Ribo-TK, Ad-PEPCK-TK or Mock;

FIG. 4 are analysis results for RNA and base sequences of trans-splicing molecules (TSM) to identify the trans-splicing action of ribozymes, after Hep3B, SKOV3 and IMR90 cell lines are infected with Mock, PL, PRT and CRT;

FIG. 5 shows results of PCR analysis to confirm distribution of PRT recombinant adenovirus DNAs after Hep3B, SKOV3 and IMR90 cell lines are infected with Mock, PL, PRT and CRT;

FIG. 6 is a graph showing evaluation of anti-hepatocelluar carcinoma effects of PRT, CRT and PL recombinant adenoviruses by tumor xenograft (wherein compared to PL/GCV (1-way ANOVA), “**” means P<0.005; “***” means P<0.0001);

FIG. 7 is a graph showing carcinoma weights of CRT, PRT, and PL recombinant adenovirus in 20 days after tumor xenograft tests using human uterine cervical cancer HeLa cells (average tumor weight and standard deviation are represented by error bars);

FIG. 8(a) is a microscopic image (magnification 40×) of H&E-stained liver and tumor tissues to confirm selective expression behaviors by Ad-PEPCK-LacZ (PL) and Ad-PEPCK.Ribo-LacZ (PRL) recombinant adenoviruses in peritoneal carcinomatosis models and FIG. 8(b) is a microscopic image (magnification 40×) of the liver and tumor tissues after β-galactosidase expression (More specifically, (a) expression behaviors of infected genes in Ad-PEPCK-LacZ-infected tissues, (b) expression behaviors of infected genes in Ad-PEPCK.Ribo-LacZ-infected tissues);

FIG. 9 is a graph showing β-galactosidase activity of normal liver, stomach and intestinal tissues, and three hepatocarcinoma tissue nodules, for PL and PRL recombinant adenovirus peritoneal carcinomatosis models;

FIG. 10(a) is a graph showing levels of hTERT mRNA expressed in tumor tissues for PL and PRL recombinant adenovirus-infected peritoneal carcinomatosis models, and FIG. 10(b) is images illustrating results of immunohistochemical staining using anti-hTERT;

FIG. 11(a) is a microscopic (×200) images of liver tissues paraffin-embedded and H&E stained on the 2^{nd}, 7^{th} and 14^{th} days, after injection of Ad-PEPCK-LacZ (PL), Ad-PEPCK.Ribo-TK (PRT) and Ad-PEPCK-TK (PT) into normal mice and addition of ganciclovir thereto, and FIG. 11(b) is a graph showing levels of liver enzymes, AST and ALT;

FIG. 12(a) is a microscopic image of the abdominal cavity of the peritoneal carcinomatosis mice, 2.5 weeks after injection of PL, PRT and CRT, and FIG. 12(b) is a microscopic image of extracted liver tissues and intraperitoneal tumor burdens.

FIG. 13 is a graph showing the weight of the tumor 2.5 weeks after administration of Hep3B cell lines, and the weight of the tumor 2.5 weeks after administration of PL, PRT and CRT (for each group, 10 mice were treated; and the weight average of the tumor together with standard deviation are represented);

FIG. 14(a) is RNA expression patterns of PL, PRT adenoviruses injected into normal livers (L) and several individual nodules of hepatocellular carcinomas (T), and FIG. 14(b) shows base sequence analysis results of trans-splicing molecules (TSM) generated from hepatocellular carcinomas in mice PRT-injected mice;

FIG. 15 is a microscopic image of livers extracted from intrahepatic multiple hepatocarcinoma mouse models on the 10th day after Mock and PRT adenoviruses were injected;

FIG. 16 are optical microscopy images of intrahepatic multiple hepatocarcinoma mouse models into which Mock and PRT are administered and paraffin blocks of liver tissues are H&E stained; and

FIG. 17 is a graph showing tumor weight of intrahepatic multiple hepatocarcinoma mouse models after administration of Mock and PRT.

### BEST MODE

Hereinafter, the present invention will be illustrated in more detail.

The term “promoter” as broadly used herein refers to a nucleotide sequence which regulates expression of another nucleotide sequence operably linked thereto in specific host cells. The term “operably linked” as used herein refers to a state wherein one nucleotide fragment is functionally linked to another nucleotide fragment and functions or expression thereof is thus affected by the another nucleotide fragment.

The term “adenovirus” as used herein has the same meaning as an adenovirus vector, which refers to a virus of the family adenoviridae. The adenoviridae includes all animal adenoviruses of genus Mastadenovirus. In particular, human adenoviruses include A-F subgenera and serotypes thereof. A-F subgenera includes, but is not limited to, human adenoviruses types 1, 2, 3, 4, 4a, 5, 6, 7, 8, 9, 10, 11 (Ad11A and Ad11P), 12, 13, 14, 15, 16, 17, 18, 19, 19a, 20, 21, 22, 23, 24, 25, 26, 27, 28, 29, 30, 31, 32, 33, 34, 34a, 35, 35p, 36, 37, 38, 39, 40, 41, 42, 43, 44, 45, 46, 47, 48 and 91.

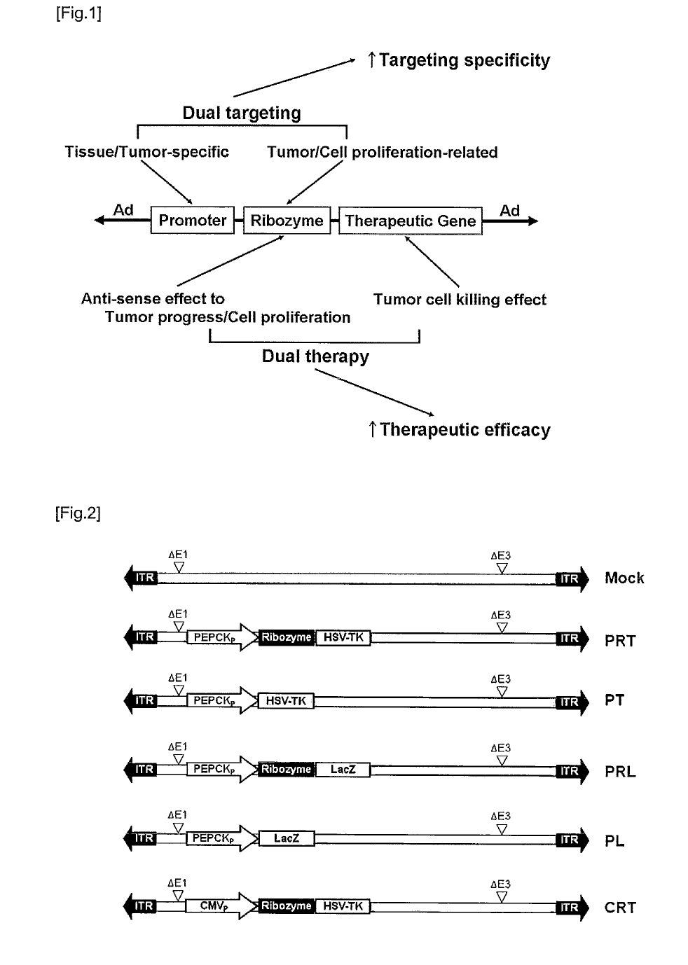

FIG. 1 is a schematic diagram illustrating recombinant adenovirus according to the present invention.

Referring to FIG. 1, the recombinant adenovirus of the present invention comprises: a tissue-specific promoter; a trans-splicing ribozyme sequence having RNA substitution activity for tumor-specific genes; and a therapeutic gene (or a reporter gene) linked to the 3′ exon of the ribozyme.

Due to the tissue-specific promoter, ribozyme is expressed only in specific target tissues, and the action of the trans-splicing ribozyme having RNA substitution activity for tumor-specific genes allows the ribozyme to exhibit trans-splicing activity only in cancer tissues, rather than normal tissues. Accordingly, the adenovirus cannot operate in other tissues except for cancer tissues, thus significantly reducing adverse effects of gene therapy and exhibiting excellent therapeutic efficacy, thereby solving the problems of superior tissue specificity as well as high efficacy which were difficult to realize in previous gene treatments.

The recombinant adenovirus of the present invention expresses trans-splicing ribozymes acting on tumor-specific RNA and therapeutic genes (or reporter genes). For this purpose, a tissue-specific promoter is operably linked to the trans-splicing ribozyme. Accordingly, the tissue-specific promoter has an influence on the expression of the trans-splicing ribozyme and regulates the expression. Preferably, the recombinant adenovirus further comprises an enhancer of the same tissue specificity as the above tissue-specific promoter.

Any promoter or enhancer may be used so long as it is a tissue-specific promoter or enhancer capable of inducing expression of target genes in a specific tissue. Representative examples of useful promoters or enhancers include liver tissue—specific PEPCK (phosphoenolpyruvate carboxykinase) genes, apolipoprotein E genes, serum albumin genes; hepatoma-specific AFP (alphafetoprotein) genes; colorectal cancer specific CEA (carcinoembryonic antigen) genes; and prostate tumor-specific PSA (prostate-specific antigen) gene promoters or enhancers. Most preferably, PEPCK gene promoters and enhancers (Roesler, W J, J. Biol. Chem. 267:21235-21243, 1992) which exhibit tissue-specificity, most clearly expressed in the liver may be used.

The promoter or enhancer may be prepared by a method well-known in the art, for example, by performing PCR using the human genome as a template and a suitable primer or using a DNA autosynthesizer.

The trans-splicing ribozyme acting on the tumor-specific RNAs are expressed only in specific tissues by tissue-specific promoters. The ribozyme expressed only in the specific tissue mediates trans-splicing reactions targeting tumor-specific genes expressed in cells and ligates therapeutic genes or reporter genes. Based on the fore-going, tumor-specific genes are modified, selectively inducing death of only cancer cells or diagnosing the same, to treat or diagnose the cancer cells.

The tumor-specific gene refers to a gene which is specifically expressed in cancer cells. Representative examples of useful tumor-specific genes include hTERT (human telomerase reverse transcriptase) mRNAs, AFP (alphafetoprotein) mRNAs, CEA (carcinoembryonic antigen) mRNAs, PSA (prostate-specific antigen) mRNAs, CKAP2 (cytoskeleton-associated protein 2) mRNAs, and the like.

Any ribozyme may be used so long as it can perform trans-splicing reactions targeting tumor-specific genes to link new therapeutic genes (or reporter genes) to the tumor-specific genes. Examples of useful ribozymes include hTERT targeting trans-splicing Group I ribozymes that are known to identify and trans-splice representative tumor-specific RNA transcripts, hTERT (human telomerase reverse transcriptase) mRNAs.

The trans-splicing activity of ribozymes expressed by promoters enables therapeutic genes to be spliced to tumor-specific genes, thus enabling treatment of cancer cells. The term “therapeutic gene” as used herein refers to a nucleotide sequence which is expressed in cancer cells and exhibits therapeutic efficacy. Examples of therapeutic genes include, but are not limited to, drug sensitizing genes, proapoptotic genes, cytostatic genes, cytotoxic genes, tumor suppressor genes, antigenic genes, antiangiogenic genes, cytokine genes and the like.

The drug sensitizing gene refers to a gene of an enzyme which converts a nontoxic prodrug into a toxic substance, which is also referred to as a suicide gene, because cells into which the gene is introduced die. That is, when prodrugs, non-toxic in normal cells, are systemically introduced, they are converted into toxic metabolites only in cancer cells to vary sensitivity for drugs and thereby to kill the cancer cells. Representative examples of useful drug-sensitive genes include HSV-tk (herpes simplex virus-thymidine kinase) genes and ganciclovir, Escherichia coli cytosine deaminase (CD) genes and 5-fluorocytosine (5-FC).

The proapoptotic gene refers to a nucleotide sequence which is expressed to induce programmed cell death. Proapoptotic genes well known to those skilled in the art include, p53, adenovirus E3-11.6K (derived from Ad2 and Ad5) or adenovirus E3-10.5K (derived from Ad), adenovirus E4 genes, p53 pathway genes and caspase-coding genes.

The cytostatic gene refers to a nucleotide sequence which is expressed in cells to stop a cell cycle. Representative examples of cytostatic genes include p21, retinoblastoma genes, E2F-Rb-fused protein genes, cyclin-dependent kinase inhibitor-coding genes (e.g., p16, p15, p18 and p19), growth arrest specific homeobox (GAX) genes (PCT Publication Nos. WO 97/16459 and WO 96/30385), and the like.

The cytotoxic gene refers to a nucleotide sequence which is expressed in cells to exhibit toxicity. Examples of cytotoxic genes include nucleotide sequences coding Pseudomoas exotoxins, lysine toxins, diphtheriae toxins and the like.

The tumor suppressor gene refers to a nucleotide sequence which is expressed in target cells to inhibit tumor phenotypes or induce cell death. Representative examples of tumor suppressor genes include tumor necrosis factor-□ (TNF-□), p53 genes, APC genes, DPC-4/Smad4 genes, BRCA-1 genes, BRCA-2 genes, WT-1 genes, retinoblastoma genes (Lee et al., Nature, 329,642, 1987), MMAC-1 genes, adenomatous polyposis coil proteins (Albertson et al. U.S. Pat. No. 5,783,666), DCC (deleted in colorectal carcinoma) genes, MMSC-2 genes, NF-1 genes, ENT tumor suppressor genes arranged in chromosome 3p21.3 (Cheng et al. Proc. Nat. Acad. Sci., 95,3042-3047, 1998), MTS1 genes, CDK4 genes, NF-1 genes, NF-2 genes and VHL genes.

The antigenic gene refers to a nucleotide sequence which is expressed in target cells to produce cell-surface antigen proteins identified in an immune system. Examples of antigenic genes well-known to those skilled in the art include carcinoembryonic antigens (CEA) and p53 (Levine, A., PCT Publication No. WO 94/02167).

The cytokine gene refers to a nucleotide sequence which is expressed in cells to produce cytokine. Representative examples of cytokine genes include GM-CSF, interleukins (IL-1, IL-2, IL-4, IL-12, IL-10, IL-19 and IL-20), interferon a, β and γ (interferon a-2b) and fusants such as interferon a-2a-1.

The anti-angiogenic gene refers to a nucleotide sequence which is expressed in cells to release anti-angiogenic factors to the outside of the cells. Examples of anti-angiogenic genes include angiostatin, vascular endothelial growth factor (VEGF) inhibitors, endostatin and the like.

In addition, through the ribozyme activity, reporter genes may be spliced to tumor-specific genes. The reporter genes spliced to tumor-specific genes in specific tissues are expressed as reporter proteins according to transcription activity of promoters. By measuring activity or amount of the expressed reporter proteins, cancer cells can be diagnosed.

The reporter gene may be selected from those well-known in the art, and may be a coding gene of LacZ, chloramphenicol acetyl transferase (CAT), renila luciferase, firefly luciferase, red fluorescent proteins (RFP), green fluorescent proteins (GFP), secreted placental alkaline phosphatase (SEAP) or herpes simplex virus-thymidine kinase (HSV-tk).

The activity of reporter proteins may be evaluated by a method well-known in the art:

Firefly luciferase (See. de Wet J. et al., Mol. Cell Biol., 7, 725-737, 1987); Renilla luciferase (See. [Lorenz W. W. et al., PNAS 88, 4438-42, 1991); chloramphenicol acetyl transferases (See. Gorman C. et al., Mol. Cell Biol., 2, 1044-1051, 1982); LacZ (See. Hall C. V. et al., J. Mol. Appl. Genet ., 2,101-109, 1983), human growth hormones (See. Selden R. et al., Mol. Cell Biol., 6, 3173-3179, 1986), green fluorescent proteins (See. Chalfie M. et al., Science, 263, 802-805, 1994) and secretory placenta alkaline phosphatase (See. Berger, J. et al., Gene, 66, 1-10, 1988). In addition, when the reporter protein is thymidine kinase, a positron emission tomography (PET) imaging method may be used.

The recombinant adenovirus of the present invention may be obtained by splicing tissue-specific promoters; trans-splicing ribozyme sequences acting on tumor-specific genes; and therapeutic genes or reporter genes, and injecting the resulting products into adenoviruses wherein E1 and E3 genes are deleted, in accordance with a method known to those skilled in the art.

In one embodiment, liver-specific PEPCK (phosphoenolpyruvate carboxykinase) gene enhancers and promoters; and hTERT RNA-specific trans-splicing ribozyme Rib21AS and HSV-tk (Herpes simplex virus-thymidine kinase) genes or Lacz genes are introduced into adenoviruses wherein E1 and E3 genes are removed, to prepare recombinant adenovirus Ad-PEPCK.Ribo-TK (Seq. No. 1) and Ad-PEPCK.Ribo-LacZ (Seq. No. 2). The adenoviruses used herein were derived from human adenovirus type 5 serotypes.

The PEPCK gene promoter may have a base sequence represented by Seq. No. 3, and the PEPCK gene enhancer may have a base sequence represented by Seq. No. 4. In addition, the Rib21AS sequence may have a base sequence represented by Seq. No. 5 and the LacZ gene may have a base sequence represented by Seq. No. 7.

Furthermore, the HSV-tk gene may have a base sequence represented by Seq. No. 6, and may be selected from those registered in genbank Reg. Nos. AAP13943, P03176, AAA45811, P04407, Q9QNF7, KIBET3, P17402, P06478, P06479, AAB30917, P08333, BAB84107, AAP13885, AAL73990, AAG40842, BAB11942, NP_{—}044624, NP_{—}044492, CAB06747, etc.

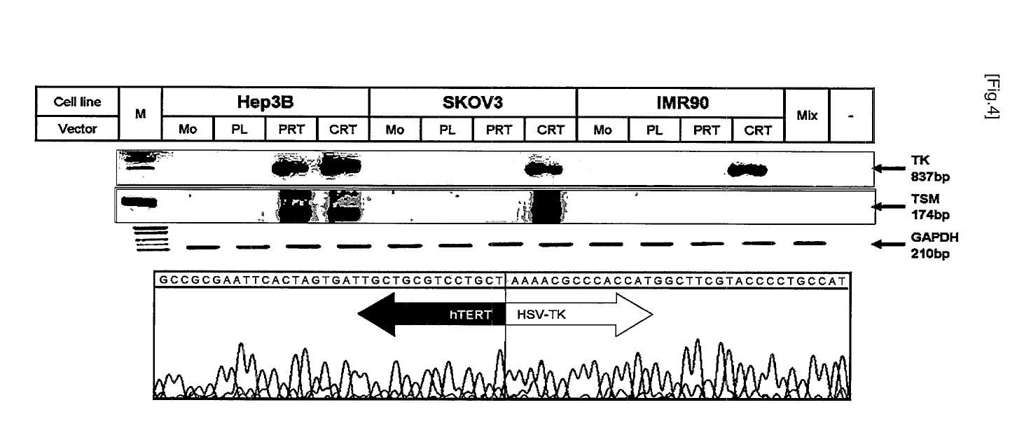

Ad-PEPCK.Ribo-TK PRT, the recombinant adenovirus of the present invention, induces high rates of cell death in hepatocellular carcinoma cells Hep3B, but does not induce cell death for ovary adenocarcinoma cells, SKOV3, rather than liver cell lines, which indicates high tissue-specificity. In addition, Ad-PEPCK.Ribo-TK PRT does not induce cell death in normal liver cell lines, THLE3, which indicates tumor-specificity (See FIG. 3).

In addition, the tissue-specific promoter-free CRT (Ad-CMV.Ribo-TK) ribozymes are expressed both in hepatocellular carcinoma cells Hep3B and in ovary adenocarcinoma cells SKOV3, to form trans-spliced molecules (TSM). Meanwhile, trans-spliced molecules (TSM) are not generated in PRT-infected ovary adenocarcinoma cells SKOV3 and normal lung embryo fibroblast cells IMR90, but are generated only in hepatocellular carcinoma cells Hep3B. These behaviors demonstrate that selective death of hepatocarcinoma cells derived from PRT is due to specific and highly accurate trans-splicing actions of hTERT RNAs of ribozymes which are selectively expressed in liver tissues (See. FIG. 4).

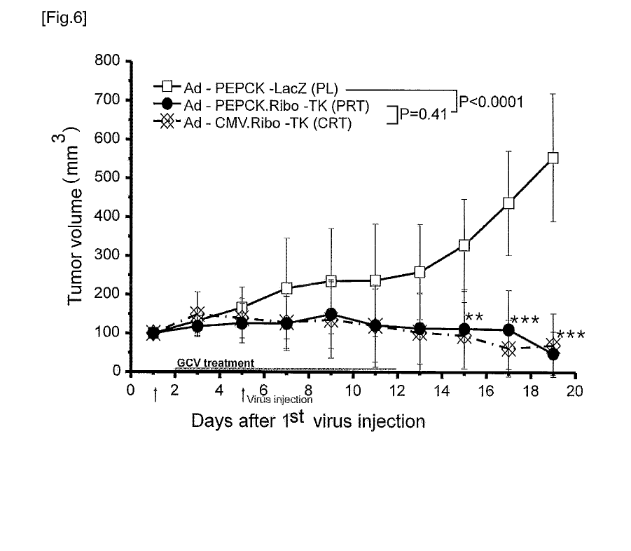

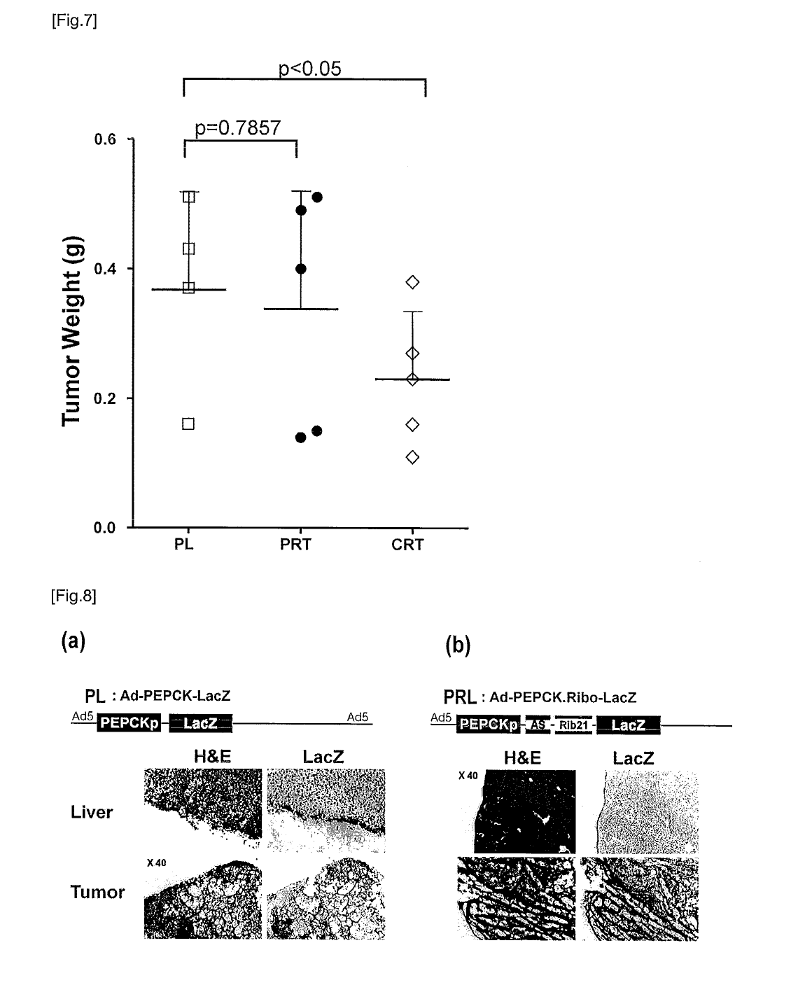

PRT was injected in combination with ganciclovir into tumor xenografted hepatomas and the volume of tumors was measured. As a result, Hep3B carcinomas were dramatically degraded, which indicates that the recombinant adenovirus exhibits anti-hepatocarcinoma (anti-HCC) activity (See FIG. 6). Meanwhile, PRT was injected in combination with ganciclovir into tumor xenografted-cervix adenocarcinoma and the weight of HeLa tumors was measured. As a result, there was not significant variation in tumor size, unlike the case wherein the administration of tissue-specific promoter-free CRT causes a 40% decrease (See FIG. 7).

PRL was administered into peritoneal carcinomatosis model mice. As a result, hTERT mRNA significantly decreased and the amount of hTERT proteins also significantly decreased (See FIG. 10). This means that owing to the recombinant adenovirus of the present invention, ribozyme efficiently performs trans-splicing reaction targeting tumor-specific genes, hTERT RNAs.

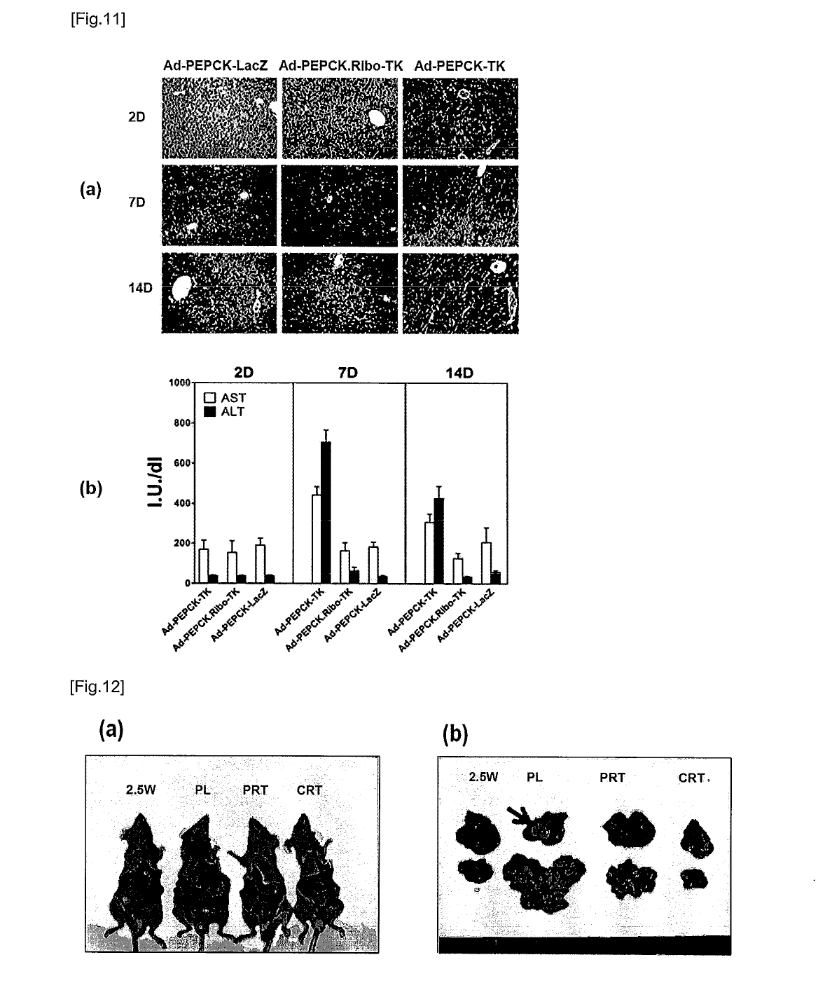

In addition, the recombinant adenoviruses were administered into normal mice and safety thereof was then evaluated. As a result, for PT-administered mice, death of liver cells and inflammation, and an increase in liver enzyme levels were observed in liver-tissues, while, for PRT-administered mice, the levels of liver tissues and enzymes were normal (See FIG. 11).

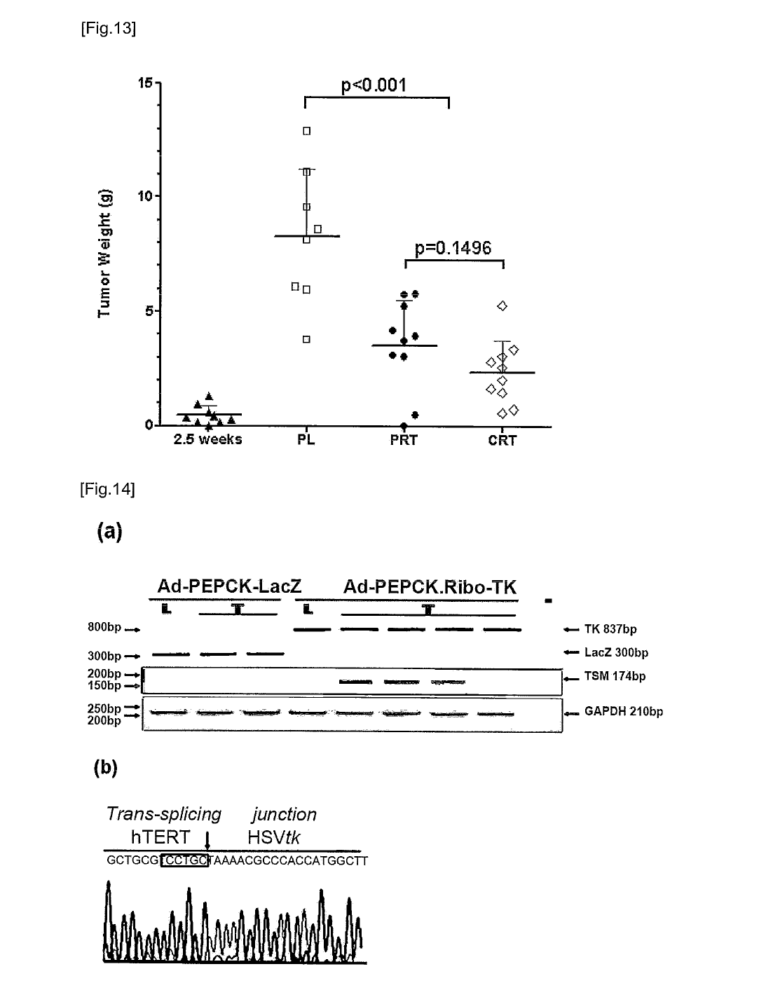

The recombinant adenoviruses were injected into peritoneal carcinomatosis model mice. As a result, tumors did not remain or were only quite small in the liver of mice wherein PRT was administered in combination with ganciclovir (See FIG. 12). In addition, PRT administration resulted in significant decrease in hepatocarcinoma weight (See FIG. 13).

RNA analysis results for the hepatocarcinoma and normal liver tissues in PRT-administered peritoneal carcinomatosis mouse models ascertained that ribozymes (TK RNA) are formed both in hepatocarcinoma and in normal liver tissues, but trans-spliced molecules (TSM) were formed only in hepatocarcinoma. In addition, from base sequence analysis of trans-spliced molecules (TSM), it could be confirmed that PRT allows therapeutic genes, HSV-tk RNA to be suitably spliced to tumor-specific genes, hTERT RNAs (See FIG. 14).

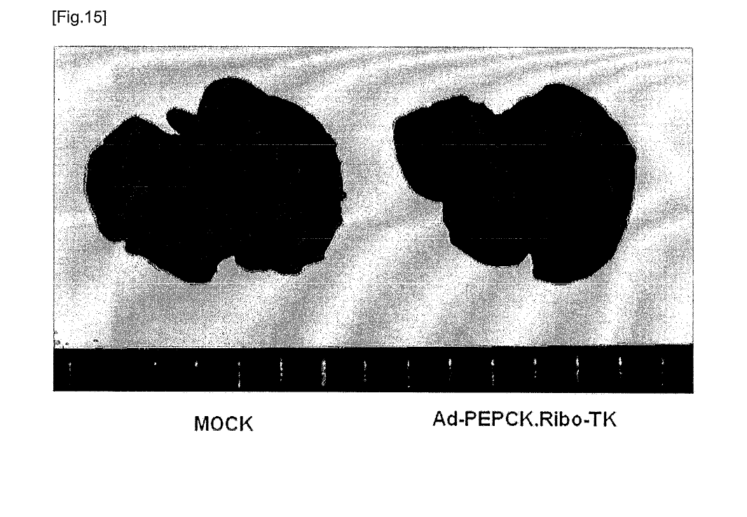

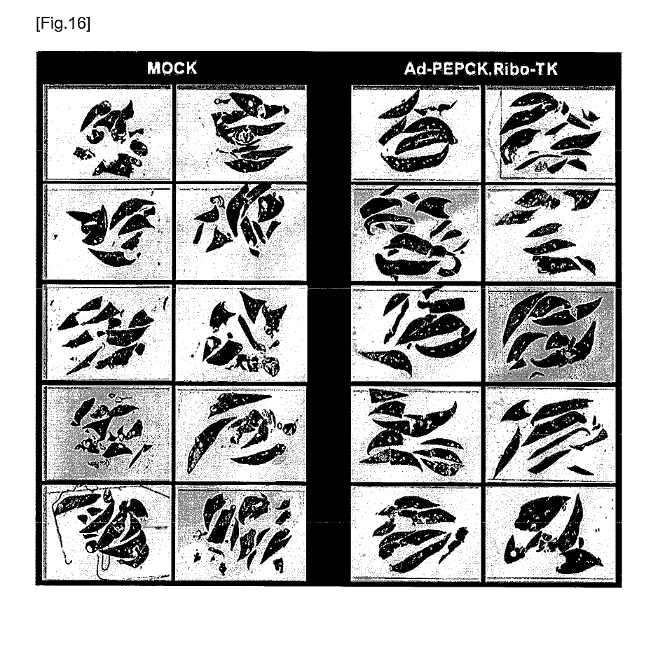

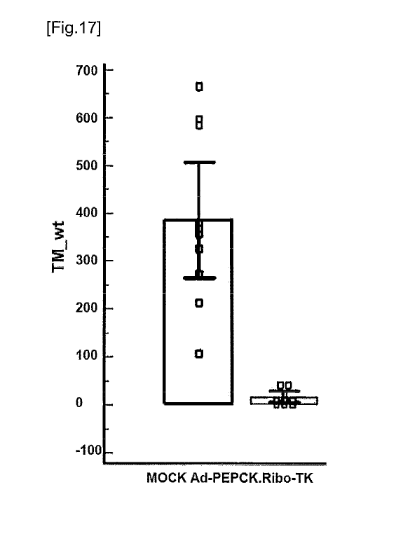

In addition, PRT together with ganciclovir was administered to intrahepatic multiple hepatocarcinomas mouse models and the livers were then extracted from the mice. As a result, tumor nodules were hardly observed (See FIG. 15). From hematoxylin-eosin (H&E) staining tumors, tissue findings wherein tumors were not observed or hepatocarcinomas were rarely observed were obtained (See FIG. 16). PRT administration caused a significant decrease in weight of hepatocarcinomas, compared to control groups (mock) (See FIG. 17). These results indicated that recombinant viruses of the present invention efficiently induce death of hepatocarcinoma tissues in vivo, thus being pharmaceutically useful for anti-cancer preparations.

As such, the recombinant adenoviruses of the present invention are efficiently transduced into human cancer cells and expressed to modify tumor-specific genes into therapeutic genes and thereby to induce expression of genes. As a result, cell death of only cancer cells can be induced.

Accordingly, the present invention provides an anticancer pharmaceutical composition comprising the recombinant adenoviruses as an active ingredient.

The anticancer pharmaceutical composition may be formulated for administration, while comprising one or more pharmacologically acceptable carriers, in addition to the active ingredient.

The pharmacologically acceptable carriers contained in the composition of the present invention may be selected from those commonly used for formulations. For compositions formulated in the form of liquid solutions, pharmacologically acceptable carriers are selected from those suitable for sterilization and human body, and examples thereof include saline, sterile water, Ringer's solution, buffered saline, albumin injections, dextrose solutions, malto-dextrine solutions, glycerol, ethanol and combinations thereof. If necessary, the composition may further comprise other general additives such as antioxidants, buffers and bacteriostatics. Furthermore, by further adding diluents, dispersants, surfactants, binders and lubricants, the composition can be formulated in the form of preparations for injection such as solutions, suspensions or emulsions, pills, tablets, capsules or granules. The carriers may be bound to target organ-specific antibodies or other ligands so that they can specifically act on the target organ.

Since the recombinant adenovirus contained in the composition of the present invention exhibits anti-tumor efficacy for a variety of tumor cells, the pharmacological composition is suitable for use in treatments of various diseases or disorders associated with tumors, for example, brain cancers, stomach cancers, lung-cancers, breast cancers, ovarian cancers, liver cancers, bronchial cancers, nasopharyngeal cancers, laryngeal cancers, esophageal cancer, pancreatic cancer, prostatic cancers, large intestine cancers, colon cancers, bone cancers, skin cancers, thyroid cancers, parathyroid gland cancers, ureter cancers, uterine cervical cancers and the like.

The pharmacological composition of the present invention may be parenterally administered and examples of parenteral administrations include, but are not limited to, intravenous, intraperitoneal, intratumoral, intramuscular, subcutaneous or local administrations. For example, when the composition is administered into the celiac for ovarian cancers and is administered into the hepatic portal vein for liver cancers, injection administration may be used; for breast and craniocervical cancers, the composition may be directly administered by injection into the tumor mass; for colon cancers, the composition may be directly administered by enema; and for bladder cancers, the composition may be directly administered into a catheter.

A dose of the anticancer pharmaceutical composition of the present invention is controlled depending on various factors, including type of disease, severity of disease, type and content of active ingredients and other ingredients of composition, type of formulation, age, body weight, health conditions, gender and diets of patients, dosage time, dosage route, secretion ratio of composition, treatment period and medications administered in conjunction therewith. However, for desired effects, the pharmaceutical composition of the present invention comprises 1×10⁵ to 1×10¹⁵ PFU/ml of recombinant adenoviruses and is typically administered in a dose of 1×10¹⁰ PFU once every two days for 5 days.

The pharmaceutical composition of the present invention may be used singly or in combination with sub-treatments such as surgical operations. Examples of chemotherapeutic agents used in combination with the composition include cisplatin, carboplatin, procarbazine, mechlorethamine, cyclophosphamide, ifosfamide, melphalan, chlorambucil, bisulfan, nitrosourea, dactinomycin, daunorubicin, doxorubicin, bleomycin, plicomycin, mitomycin, etoposide, tamoxifen, taxol, transplatinum, 5-fluorouracil, vincristin, vinblastin, methotrexate and the like. Radiation therapies used in combination with the composition are X- and □-ray radiations. Preferably, the composition may be used in combination with ganciclovir.

The recombinant adenoviruses of the present invention are efficiently infected into human cancer cells and are expressed, thus splicing reporter genes to tumor-specific genes and thereby allowing selectively identification of cancer cells.

Accordingly, the present invention also provides a composition for cancer diagnosis comprising the recombinant adenoviruses as an active ingredient.

The present invention also provides a cancer imaging method comprising S1) introducing the recombinant adenoviruses into cancer cells; and S2) detecting reporter proteins from the cancer cells.

Since the recombinant adenoviruses of the present invention allow host cells to express reporter proteins, recombinant adenovirus-transduced cancer cells can express reporter proteins. The detection of the reporter proteins may be carried out in accordance with the afore-mentioned method well-known in the art.

Cancers or tumors that can be diagnosed by the present invention are not particularly limited, and preferred examples thereof include stomach cancers, lung cancers, breast cancers, ovarian cancers, liver cancers, bronchial cancers, nasopharyngeal cancers, laryngeal cancers, pancreatic cancer, bladder cancers, large intestine cancers, colon cancers, uterine cervical cancers, brain cancers, prostatic cancers, bone cancers, skin cancers, thyroid cancers, parathyroid gland cancers, ureter cancers and the like. Most preferred are liver cancers.

In one embodiment, liver-tissue specific PEPCK (phosphoenolpyruvate carboxykinase) gene enhancers and promoters; and hTERT RNA-specific trans-splicing ribozyme Rib21AS and LacZ genes are introduced into Type 5 adenoviruses, wherein E1 and E3 genes are deleted, to manufacture Ad-PEPCK.Ribo-LacZ (Seq. No. 2).

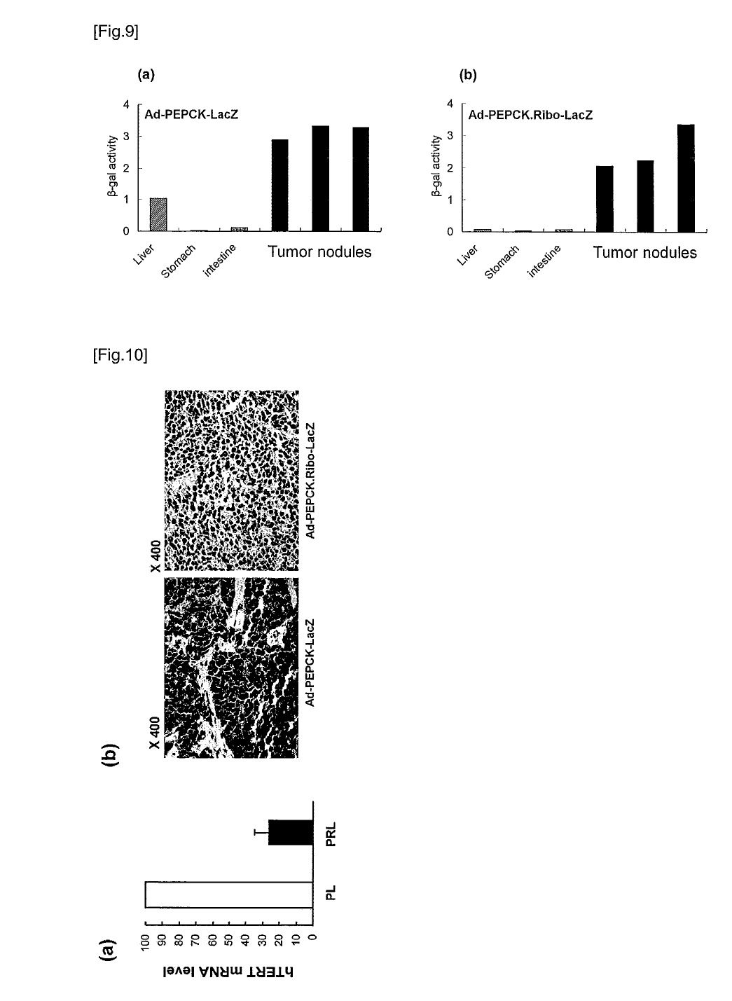

For the Ad-PEPCK.Ribo-LacZ(PRL)—introduced peritoneal carcinomatosis mouse models, lacZ was expressed only in tumor tissues, but was not expressed in normal liver surfaces. Conversely, for recombinant viruses Ad-PEPCK-LacZ (PL) containing no ribozymes acting on tumor-specific genes, lacZ was expressed both in normal liver tissues and in tumor tissues (See FIG. 8). Furthermore, both PL and PRL exhibited β-galactosidase activity only in the liver owing to liver tissue-specific PEPCK promoters, but PRL exhibited β-galactosidase activity only in tumor tissues due to ribozyme acting on tumor cell-specific genes (See FIG. 9). These results indicate that the recombinant adenovirus of the present invention is useful for in vivo cancer cell diagnosis.

### Mode for Invention

Hereinafter, exemplary examples will be provided for a further understanding of the invention. The following examples are for illustrative purposes only and are not intended to limit the scope of the present invention.

### EXAMPLE 1

### Preparation of Recombinant Adenoviruses Comprising Tissue-Specific Promoters, Ribozymes Acting on Tumor-Specific Genes and Therapeutic Genes or Reporter Genes

In order to manufacture recombinant adenoviruses comprising tissue-specific promoters and ribozymes acting on tumor-specific genes, ribozymes were manufactured by a method well-known in the art (Kwon et al., Mol. Ther. 12:824-834, 2005).

PEPCK gene enhancers and promoters were manufactured by a method disclosed in known literatures (Kwon, B. S. at al, Specific regression of human cancer cells by ribozyme-mediated targeted replacement of tumor-specific transcript. Mol Ther 12, 824-834, 2005; Song, M. S. & Lee, S. W. Cancer-selective induction of cytotoxicity by tissue-specific expression of targeted trans-splicing ribozyme. FEBS Lett 580, 5033-5043, 2006)

That is, Rib21AS ribozymes targeted at U21 on hTERT RNA were generated to contain extended internal guide sequence (IGS) such as an extended P1 helix, an additional 6-nt-long P10 helix and 325-nt-long antisense sequence complementary to the downstream region of the targeted hTERT RNA uridine. cDNA as a 3′ exon encoding bacterial β-galactosidase (lacZ) or herpes simplex virus thymidine kinase (HSV-tk) gene was inserted at the NruI/XbaI cleavage enzyme site which is present at the downstream region of the modified Group I intron-expressing structures. The resulting ribozyme was then cloned into pcDNA or pPEPCK-LCR. The resulting ribozyme cDNA flanked by promoter and 3′ exon sequence was cloned into SpeI/BstBI site of pAdenoVator-CMV5-IRES-GFP shuttle vector (Qbiogene). Recombinant adenovirus vectors encoding the ribozymes were then generated using the in vivo homologous recombination technique in Bacteria (BJ5183) as follows. Briefly, the shuttle plasmid was linearized with PmeI, and then cotransformed into BJ5183 cells with an E1/E3 deleted adenoviral type5 backbone genome (pAdenoVator ΔE1/E3, Qbiogene). Recombinant vectors generated by homologous recombination in BJ5183 cells were isolated, and linearized with Pacl. The linearized vectors were then infected into 293 cells, and the produced recombinant adenoviruses were isolated through three rounds of plaque purification. The final product, recombinant adenovirus, was amplified, separated, concentrated using Vivapure AdenoPACK™ 100 (Sartorius A G, Edgewood, N.Y.) and was then quantitatively measured by a TCID50 method.

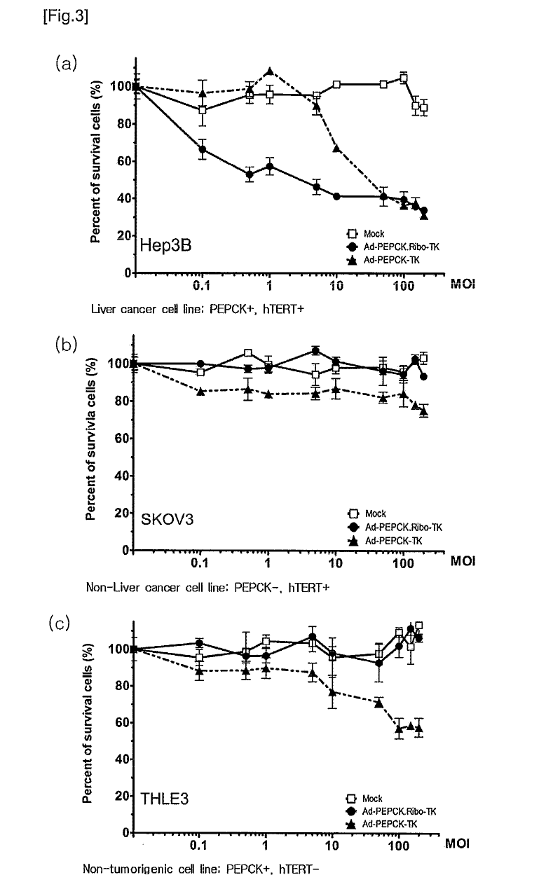

FIG. 2 illustrates a gene map of the recombinant adenovirus vector thus manufactured.

Referring to FIG. 2, MOCK refers to an adenovirus containing no foreign gene, Ad-PEPCK.Ribo-TK (or PRT) refers to a recombinant adenovirus encoding the specific Rib21AS ribozyme with HSV-tk gene under the control of liver-specific PEPCK promoter, Ad-PEPCK-TK (or PT) refers to a recombinant adenovirus which expresses HSV-tk genes under the control of liver-specific PEPCK promoters, Ad-PEPCK.Ribo-LacZ (or PRL) refers to a recombinant adenovirus encoding the specific Rib21AS ribozymes with lacZ genes under the control of liver-specific PEPCK promoters, Ad-PEPCK-LacZ (or PL) refers to a recombinant adenovirus encoding lacZ gene under the PEPCK promoter, and Ad-CMV.Ribo-TK (or CRT) refers to a recombinant adenovirus encoding the Rib21AS ribozyme with HSV-tk gene under the strong constitutional CMV(cytomegalovirus immediate early) promoter.

### EXAMPLE 2

### Cytotoxicity Test

### 2-1. Cell Culture

The cell lines used herein are available from ATCC (American Type Culture Collection), and are as follows:

SKOV3 (Human ovary adenocarcinoma cells); HeLa (Human cervix adenocarcinoma cells); Hep3B and HepG2 (Human Hepatocellular carcinoma cells); IMR90 (telomerase-free Normal human lung embryo fibroblast); and THLE3 (SV40 large T antigen immortalized primary normal liver cells).

Of these cell lines, SKOV3 and HeLa were maintained in Dulbecco's modified Eagle's medium (DMEM) with 10% heat-inactivated fetal bovine serum(FBS; Jeil Biotech Services Inc., Seoul, Korea), 50 U/ml penicillin G and 50 μg/ml streptomycin (Sigma, St.Louis, Mo.). HepG2, Hep3B and IMR90 cell lines were cultured in a bronchial/tracheal epithelial cell growth medium (Cambrex, East Rutherford, N.J.) containing a 10% FBS-containing EMEM solution, THLE3 cell lines were 10% FBS, 6.5 ng/ml triiodothyromine, 50 μg/ml gentamicin and 50 ng/ml amphotericin-B. These cell lines were incubated in an incubator at 37□, 5% carbon dioxide prior to the test.

### 2-2. MTS Assay

In order to identify liver tumor-specific efficacy and specificity of anticancer effects, for recombinant adenoviruses encoding ribozymes targeting hTERT under the liver tissue-specific PEPCK promoters, the cell lines were infected with Ad-PEPCK.Ribo-TK, Ad-PEPCK-TK or Mock at various multiplicities of infection (MOI) and were then treated with 100 μM ganciclovir. The cell viability was assayed by MTS. The results thus obtained are represented as “mean±standard deviation” for three tests.

The Cell proliferation (MTS) assay was employed using standard protocols with minor modifications. Specifically, cells were seeded in 96-well plate at 5×10³ cells/well and were then incubated overnight at 37□. Units of three wells were infected (three times repetition) with the adenoviruses manufactured at various multiplicities of infection (MOI).

At one day post-infection, 100 μM ganciclovir (GCV, Cymevene, Roche, Basel, Switzerland) was added to each plate and the cells were further incubated for 5 days. Subsequently, twenty μl of CellTiter 96Aqueous one solution reagent (MTS, Promega) in 100 μl of Opti-MEM were then added to each well, and incubated for 1 to 4 hours, based on the rate of color change.

Measuring absorbance at a wavelength of 490 nm was used to assess cell viability. The cell viability after treatment of GCV was determined by an absorbance ratio with respect to an absorbance measured from GCV-untreated cells. Based on 100% of the cell viability measured from Mock-infected cells, the cell viability for the remaining samples was recalculated.

After treatment of recombinant adenoviruses at various multiplicities of infection, various cell lines including hTERT+ hepatocellular carcinoma cell lines (Hep3B), hTERT+ ovary adenocarcinoma cell lines (SKOV3) rather than hTERT+ liver cancers, and hTERT− nomal liver cell lines (THLE3) were tested. The results thus obtained are shown in FIG. 3.

Referring to FIG. 3, (a) in hepatocelluar carcinoma cell lines, Hep3B, exhibited a high death ratio of cells infected with Ad-PEPCK.Ribo-TK and Ad-PEPCK-TK, when compared to Mock, and in particular, at a low MOI, Ad-PEPCK.Ribo-TK exhibited high death levels, as compared to Ad-PEPCK-TK, and meanwhile (b) Ad-PEPCK.Ribo-TK did not induce cell death for all types of infection in ovary adenocarcinoma cell lines SKOV3 (not liver cell lines), which indicates tissue-specificity. Furthermore, (c) in non-tumorous liver cell lines, THLE3, Ad-PEPCK-TK induced more efficient cell death, as compared to Mock, and Ad-PEPCK.Ribo-TK did not induce cell death, which obviously indicates tumor-specificity.

These results indicate that (1) independent from hTERT expression, Ad-PEPCK-TK induces hepatotoxicity, but Ad-PEPCK.Ribo-TK induces hTERT+ hepatocellular carcinoma cell-specific cytotoxicity, and (2) for cytotoxicity in hepatocellular carcinoma cells Hep3, Ad-PEPCK.Ribo-TK more efficiently induces hepatotoxicity, as compared to Ad-PEPCK-TK. From the fore-going, it can be seen that Ad-PEPCK.Ribo-TK selectively and more efficiently induces expression of suicide genes in hTERT expressing hepatocellular carcinoma cells.

### EXAMPLE 3

### RNA Analysis

Hep3B, SKOV3 and IMR90 cell lines were infected at 150 MOI with Mock, PL, PRT and CRT, and RNA analysis was performed to confirm trans-splicing activity of ribozymes.

Specifically, in order to analyze ribozyme RNA levels in recombinant adenovirus infected cells or tissues from mice, a total of 5 μg of RNA was isolated using Trizol (Invitrogen, Carlsbad, Calif.) supplemented with 20 mM EDTA and reverse transcribed with an oligo(dT) primer in the presence of 10 mM L-argininamide. The cDNAs were amplified with HSV-tk specific primers (5′-GCGAACATCTACACCACACA-3′ [Seq. No. 8] and 5′-AGTTAGCCTCCCCCATCTC-3′ [Seq. No. 9]) or ITR (inverted terminal repeat)-specific primers (5′-GGAATTCTGGAGTTTGTGACGTGGCG-3′ [Seq. No. 10] and 5′-GCTCTAGATGGCCAAATCTTACTCGGTTACGC-3′ [Seq. No. 11]). For verification, the cDNAs were amplified with GAPDH specific primers (5′-TGACATCAAGAAGGTGGTGA-3′ [Seq. No. 12] and 5′-TCCACCACCCTGTTGCTGTA-3′ [Seq. No. 13]).

For the trans-spliced RNA products in cells, mouse tissues, and tumors, total RNAs were reverse transcribed with a primer specific for HSV-tk (5′-CGGGATCCTCAGTTAGCCTCCCCCAT-3′ [Seq. No. 14]) in the presence of 10 mM L-argininamide, and the resulting cDNA was amplified with a 5′ primer specific to the 5′ end of the hTERT RNA(5′-GGGGAATTCAGCGCTGCGTCCTGCT-3′ [Seq. No. 15]) and with a 3′ primer specific to the 3′ exon HSV-tk sequence (5′-GTTATCTGGGCGCTTGTCAA-3′ [Seq. No. 16]). The amplified cDNA was then reamplified with 5′ primer specific for the trans-splicing junction (5′-GCTGCGTCCTGCT AAAAC-3′ [Seq. No. 17]) and with a nested 3′ primer specific to the HSVtk sequence (5′-CAGT AGCGTGGGCATTTTCT-3′ [Seq. No. 18]), cloned, and sequenced.

Real-time Polymerase Chain Reaction (PCR) conditions are as follows:

40 cycles of 30 seconds at 95□; for 40 seconds at 55□; for one minute at 72□

We used GAPDH to control for the standard curve in the reaction mix. The threshold levels obtained from the hTERT were adjusted to the threshold levels found in the GAPDH reaction to correct for minor variation in cDNA loading. A real-time polymerase chain reactor, Rotor-gene system (Corbett, San Francisco, Calif.), was used for amplification.

FIG. 4 illustrates analysis results for RNA and base sequences of trans-splicing molecules (TSM) to identify the trans-splicing action of ribozymes, after Hep3B, SKOV3 and IMR90 cell lines were infected with Mock, PL, PRT and CRT.

Referring to FIG. 4, for CRT-infected hTERT+ Hep3B and SKOV3 cells, trans-spliced molecules (TSM, 174 bp) were generated, while, for hTERT-IMR90 cells, ribozymes (TK RNA) were generated, but TSM was not generated. On the other hand, ribozymes were selectively expressed in PRT-infected hepatocellular carcinoma cell lines Hep3B and trans-splicing molecules (TSM) were then generated only therein. In addition, TSM was not detected from RNAs extracted from a blend of mock-infected Hep3B cells and CRT-infected IMR90 cells.

In conclusion, trans-splicing molecules (TSM) of PRT-infected Hep3B cells are generated as the result of specific RNA replacement via the ribozyme-mediated liver-specific trans-splicing reaction with the target hTERT RNA. In addition, sequence analysis of the TSM verified that PRT virus accurately targeted U21 of the hTERT RNA and spliced its 3′ exon onto the target RNA in the cells as intended.

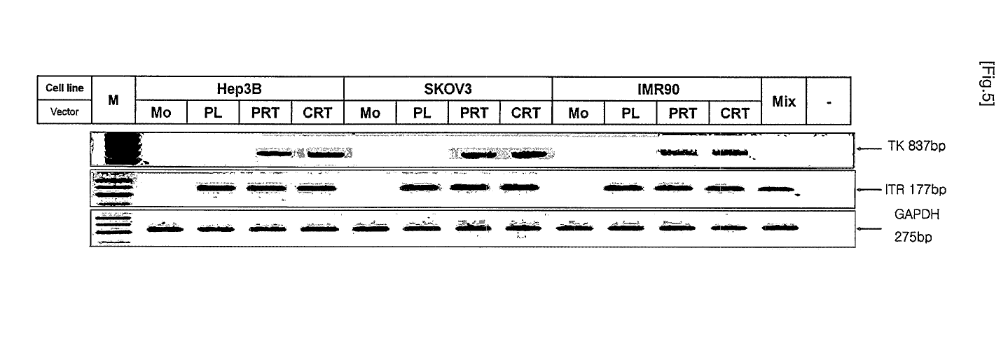

FIG. 5 shows results for PCR analysis to confirm distribution of PRT recombinant adenovirus DNAs after Hep3B, SKOV3 and IMR90 cell lines are infected with Mock, PL, PRT and CRT.

Referring to FIG. 5, DNAs of PRT recombinant adenoviruses were observed in SKOV3, IMR90 and Hep3B. This indicates that selective expression of the ribozyme in Hep3B cells was not due to specific delivery of PRT virus only into these cells.

### EXAMPLE 4

### Identification of in vivo Anticancer Activity by Tumor Xenograft

### 4-1. Test Animals

4 to 5 week-old male BALB/cAnNCrI nude mice (Orientbio, Inc., Sungnam, Korea) were used as test animals. The test animals were kept under specific pathogen-free conditions, acclimated to laboratory environment for a minimum one week prior to use and were handled in an accredited Korean FDA animal facility in accordance with AAALAC International Animal Care policies (Accredited Unit-Korea Food and Drug Administration: Unit Number-000996).

### 4-2. Tumor Xenograft Using Hep3B Cells

In order to evaluate the efficacy of PEPCK promoter-driven ribozyme expression in vivo, hTERT+ Hep3B cells were introduced into subcutaneous tissues of athymic mice to perform hepatocellular carcinoma xenograft, and PL, PRT or CRT viruses were directly injected into growing carcinomas to ascertain anti-hepatocellular carcinoma (anti-HCC) activity.

Specifically, to prepare subcutaneous tumor model in mice, 2×10⁷ Hep3B cells were injected into the flank region of male nude mice. Generally, after 3 weeks, tumor nodules (diameter: 6 to 9 mm, 140 mm³) grew. The mice were randomly assigned to two treatment groups, i.e., a treatment group (n=8) with Ad-PEPCK.Ribo-TK (PRT) and a treatment group (n=6) with Ad-CMV.Ribo-TK (CRT). The test was further performed for a control group (n=5) with Ad-PEPCK-LacZ (PL).

Viruses of 1×10⁹ pfu (plague forming unit) were injected into the tumors thus obtained and were then injected thereinto again after 5 days. The mice were treated with ganciclovir (GCV) at 50 mg/kg once per day after primary injection and were then maintained for 10 days. The tumor growth was evaluated by periodic measurement with calipers every two to three days and a tumor volume was calculated by Equation below:

Tumor volume=maximal length×(perpendicular width)²

FIG. 6 is a graph showing evaluation of anti-hepatocellular carcinoma effects of PRT, CRT and PL recombinant adenoviruses by tumor xenograft.

Referring to FIG. 6, Tumors that were injected with the control virus, PL, and treated with GCV grew continuously up to 2 cm in diameter by the end of 20^{th} day when they were euthanized. On the other hand, treatment with PRT virus/GCV or CRT virus/GCV caused a dramatic regression of the Hep3B tumors up to less than 3 mm in diameter, compared to control treatment of PL virus/GCV (As a result of ANOVA statistical analysis, the data was considered significantly different (p<0.0001)).

In particular, complete tumor regression was observed in three of mice infected with either PRT or CRT. As can be seen from the in vitro cytotoxicity analysis in Example 2, there was no statistically significant difference in tumor regression when treated with PRT virus/GCV and with CRT virus/GCV (ANOVA, p=0.41).

### 4-3. Tumor Xenograft Using HeLa Cells

In order to identify the in vivo tissue-specificity of PRT viruses, instead of liver cancer, human cervix adenocarcinoma cells HeLa cells 1×10⁷ were injected into the flank region of male nude mice to obtain tumor nodules having a diameter 6 to 9 mm after 2 weeks. The CRT, PRT and PL recombinant adenoviruses (each, n=5) were directly injected into the growing tumors. The mice were treated with ganciclovir (GCV) at 50 mg/kg once per day after the first injection and were then maintained for 10 days.

FIG. 7 is a graph showing tumor weights of CRT, PRT and PL virus/GCV treatment after 20 days in tumor xenograft tests using human cervix adenocarcinoma cells HeLa.

Referring to FIG. 7, in 20 days after the first injection of recombinant adenoviruses, CRT/GCV-treated mice showed an about 40% decrease in tumor size, as compared to a control group (ANOVA; p<0.05), but the PRT/GCV-treated Group did not undergo significant variation in uterine cervical tumor size (ANOVA; p=0.7857).

These results demonstrated that HSV-tk induction from adenoviral vector encoding hTERT-targeting trans-splicing ribozyme driven by PEPCK promoter rendered tumor growth in only hepatocarcinoma xenografts highly sensitive to the prodrug GCV, with almost the same efficiency as the ribozyme-mediated HSV-tk expression via the strong CMV promoter.

### EXAMPLE 5

### Identification of Anticancer Effects Using Peritoneal Carcinomatosis Model

### 5-1. Establishment of Peritoneal Carcinomatosis Models of Hep3B Hepatoma Cell Lines

To evaluate the specificity and efficacy of the liver-specific hTERT-targeting ribozyme in a more clinically relevant condition, human hepatocarcinoma-derived peritoneal carcinomatosis model by intraperitoneal injection of 2×10⁷ Hep3B cell into nude mice was prepared. Macroscopic and multiple Hep3B tumor nodules were formed in the various regions including liver in the mice abdomen 3 weeks after intraperitoneal tumor inoculation.

In order to analyze specific transgene induction, 1×10⁹ pfu Ad-PEPCK-LacZ (PL) or Ad-PEPCK.Ribo-LacZ (PRL) in 100 μl PBS was injected intraperitoneally twice every two days after the establishment of carcinomatosis. After 2 days, mice was sacrificed, and all abdominal organs and tumors were isolated, washed with PBS, frozen and then freeze-fragmented into slices of 20 μm thickness. The resulting slices thus obtained were used for immunohistochemicai analysis, Hematoxylin and Eosin staining (H&E staining) or β-galactosidase assay.

5-2. β-galactosidase Assay and Immunohistochemistry

Two days after final injection of viruses (Ad-PEPCK-LacZ or Ad-PEPCK.Ribo-LacZ) into intraperitoneally established carcinomatosis in mice, hTERT-dependent transgene expression was analyzed qualitatively by X-gal staining according to the manufacturer's instructions using the β-galactosidase staining kit (Invitrogen Corporation).

After sacrifice, tissues and carcinomas were isolated from the mice, sectioned and frozen in an anti-freezing solution (Sakura Finetek, Zoeterwoude, The Netherlands). 8 micrometer thick frozen sections were fixed with 100 mmol/l PBS (pH 7.4) containing 2% para-formaldehyde at room temperature for 10 minutes and were stained with β-galactosidase overnight at 37□. Then, H&E counterstaining was performed and then observed under the light microscope.

Meanwhile, removed organs from adenovirus-infected mice (about 100 mg in weight) were extracted with a lysis buffer (200 μl: 0.1 M Tris-HCl, 2 mM EDTA and 0.1% Triton X-100, pH 7.8) at room temperature for 15 minutes and were then centrifuged at 4□ at 13,000 rpm for 10 minutes. The supernatant was transferred to a 1.5 ml tube and was supplemented with 400 μl of ONPG solution (o-nitrophenyl-β-D-galactopyranoside, 120 mM Na₂HPO₄, 80 mM NaH₂PO₄, 2 mM Mg₂SO₄, 100 mM β-mercaptoethanol, 4 mg/ml ONPG, Sigma, St. Louis, Mo.). The resulting solution was reacted at 37□ for 30 minutes, the reaction was stopped with 500 μl 1M Na₂CO₃, and the reaction results were measured by a UV spectrometer (Bio-Rad, Hercules, Calif.) at a wavelength of 420 nm.

In order to measure telomerase expression in tumor nodules from the adenovirus-treated mice, the tumor tissues were fixed with formalin, wax was removed from the paraffin-embedded tumor tissues and water was added thereto. Immunohistochemistry was carried out with DAKO EnVision kits (Dako, Carpinteria, Calif.). Endogenic peroxidase was blocked by dipping sections in 3% aqueous hydrogen peroxide for 10 minutes. Antigen was retrieved with 10 min microwave treatment in 10 mmol/l citrate buffer, pH 6.0. Diluted primary antibodies (1:100) against hTERT (Santa Cruz Biotechnology Inc.) were treated for 1 h at room temperature. Sections were then incubated with the secondary antibody and avidin-biotin-peroxidase complex. The slides were slightly counterstained with hematoxylin and eosin.

FIG. 8(a) is microscopic image (magnification 40×) of H&E-stained liver and tumor tissues to confirm selective expression behaviors by Ad-PEPCK-LacZ (PL) and Ad-PEPCK.Ribo-LacZ (PRL) recombinant adenoviruses in peritoneal carcinomatosis models and FIG. 8(b) is microscopic image (magnification 40×) of the liver and tumor tissues after β-galactosidase expression.

Referring to FIG. 8, for PL-infected mice, lacZ expression was observed both in normal liver tissues and in tumor surfaces, while, for PRL, lacZ was selectively expressed only in carcinomas and was not expressed in normal liver surfaces. These results indicate that PRL may be utilized in diagnosis of in vivo hepatocarcinoma cells.

FIG. 9 is a graph showing β-galactosidase activity of normal liver, stomach and intestinal tissues, and three hepatocarcinoma tissue nodules, for PL and PRL recombinant adenovirus peritoneal carcinomatosis models.

Referring to FIG. 9, tumor-specific lacZ expression by Ad-PEPCK.Ribo-LacZ (PRL) was confirmed with analyzing β-galactosidase activity in the various tissue extracts of the mice with peritoneal carcinomatosis. By PEPCK promoters, both Ad-PEPCK-LacZ (PL) and Ad-PEPCK.Ribo-LacZ (PRL) adenoviruses exhibited β-galactosidase activity only in the liver, but Ad-PEPCK-LacZ (PL) had no tumor tissue-specific ribozyme activity and thus exhibited the activity in tumor tissues and normal liver cells, while Ad-PEPCK.Ribo-LacZ (PRL) were expressed only in tumor tissues due to tumor-specific hTERT RNA-specific ribozymes. These results indicated that Ad-PEPCK.Ribo-LacZ specifically and efficiently induces expression of infected genes in vivo.

5-3. hTERT mRNA Expression Assay Using Real-Time PCR

In order to confirm the reduction level of target RNAs in cells by recombinant adenoviruses encoding specific ribozymes, hTERT mRNA levels in tumors of Ad-PEPCK.Ribo-LacZ (PRL)-injected mice and Ad-PEPCK-LacZ (PL)-injected mice were measured using real-time PCR.

Specifically, to measure a decrease of hTERT RNAs inhibited by ribozymes in vivo, PL and PRL recombinant adenoviruses were injected into peritoneal carcinomatosis model mice, and after 2 days, complementary DNAs were amplified by real-time PCR using the total RNA 2 μg extracted from the separated hepatocarcinoma as a template.

Primers used for hTERT amplification were:

| | | |
|---|---|---|
| (1) | 5′-CGGAAGAGTGTCTGGAGCAA-3′ | [Seq. No. 19] |
| and | | |
| (2) | 5′-GGATGAAGCGGAGTCTGGA-3′ | [Seq. No. 20] |

All reagents except Taq polymerases (Takara, Otsu, Shiga, Japan) were obtained for the analysis from the SYBR-Green core reagent kit (Molecular Probes, Eugene, Oreg.). The protocol was followed as the manual of the PCR-kit [12.5 μl SYBR Green Mix, 0.2 μl cDNA, 1 μl primer pair mix (5 pmol/μl each primer), and 11.3 μl H₂O].

The polymerase chain reaction (PCR) conditions were as follows:

40 cycles of 30 sec at 95□; 40 sec at 55□; and for one min at 72□

GAPDH was used as a control for standard curve in reaction mix. The threshold levels obtained from the hTERT were adjusted to the threshold levels found in the GAPDH reaction to correct for minor variation in cDNA loading. A real-time polymerase chain reactor, Rotor-gene system (Corbett, San Francisco, Calif.) was used for amplification.

FIG. 10(a) is a graph showing levels of hTERT mRNA expressed in tumor nodules from PL and PRL recombinant adenovirus-infected peritoneal carcinomatosis models, and FIG. 10(b) is images illustrating results of immunochemical staining using anti-hTERT.

Referring to FIG. 10, for PRL treated mice, hTERT RNAs were significantly (up to 75%) decreased and, similarly, hTERT proteins were also significantly decreased. This indicates that trans-splicing ribozymes induce expression of therapeutic genes as well as reduction of target molecules, thereby improving therapeutic efficacy.

In addition, RACE RT-PCR analysis showed that all trans-splicing products generated in hepatocellular carcinoma of PRL-injected mice were obtained from reactions only with the targeted hTERT RNA. This indicates that trans-splicing ribozymes are highly target-specific in vivo.

### EXAMPLE 6

### Hepatotoxicity of Ad-PEPCK.Ribo-TK in Normal Mice

Prior to confirmation of Ad-PEPCK.Ribo-TK (PRT)-specific anti-tumor activity, Ad-PEPCK-LacZ (PL), Ad-PEPCK.Ribo-TK (PRT) and Ad-PEPCK-TK (PT) were intravenously injected into normal mice to evaluate hepatotoxicity.

Specifically, 2.5×10¹⁰ Ad-PEPCK-LacZ(PL)(n=15), Ad-PEPCK.Ribo-TK(PRT)(n=15), and Ad-PEPCK-TK(PT)(n=15) adenovirus in 100 μl buffer were injected into the tail veins of male BALB/C mice, and 50 mg virus/kg ganciclovir (GCV) were then added thereto for 10 days daily twice. After GCV addition, blood was collected from the heart of every 5 mice on the 2nd, 7th and 14th days, and liver enzymes (serum AST and ALT) were measured, sacrificed and the liver tissues were separated and subjected to histologic examination.

FIG. 11(a) is microscopic (×200) images of liver tissues paraffin-embedded and H&E stained on the 2nd, 7th and 14^{th} days, after injection of Ad-PEPCK-LacZ (PL), Ad-PEPCK.Ribo-TK (PRT) and Ad-PEPCK-TK (PT) into normal mice and addition of ganciclovir thereto, and FIG. 11(b) is a graph showing levels of liver enzymes, AST and ALT.

Referring to FIG. 11, Ad-PEPCK.Ribo-TK(PRT)-injected mice exhibited no variation in liver tissue and liver enzyme levels all through 14 days, similar to Ad-PEPCK-LacZ(PL)-injected mice. This indicates that since liver tissues of normal mice had no target hTERT RNA, they exhibited no HSV-tk activation by PRT. Meanwhile, Ad-PEPCK-TK (PT)-injected mice showed degeneration in liver cells on the 2^{nd} day, an increase in liver cell death and serious inflammation opinions on the 7^{th} day and behaviors thereof were continuously observed to the 14^{th} day (FIG. 11(a)). Ad-PEPCK.Ribo-TK(PRT)-injected mice exhibited a liver enzyme level which is comparable to Ad-PEPCK-LacZ(PL)-injected mice and similar to normal values (AST; 130˜150 IU/L, ALT; 30˜40 IU/L), but Ad-PEPCK-TK(PT)-injected mice exhibited a significant increase in lever enzyme level on the 7^{th} day (FIG. 11b).

These results indicate the fact that normal liver tissues having no target molecule do not induce any hepatotoxicity to the liver tissues (that is, normal liver tissues are safe), even though 2.5×10¹⁰ Ad-PEPCK.Ribo-TK (PRT) virus were systemically administered.

### EXAMPLE 7

### Efficient Regression of Hepatocarinoma in Peritoneal Carcinomatosis Mice Following Systemically Delivered Ad-PEPCK.Ribo-TK Plus GCV

### 7-1. Confirmation of Anti-Tumor Effects to Peritoneal Carcinomatosis

To confirm anti-tumor effects, the mice which had been established with intraperitoneal tumor after 2.5 weeks of intraperitoneal injection of 2×10⁷ Hep3B were randomized into the following groups (n=10 mice per each group): (1) Ad-PEPCK-LacZ, (2) Ad-PEPCK.Ribo-TK and (3) Ad-CMV.Ribo-TK. Then, 2.5×10¹⁰ v.p adenoviruses were intraperitoneally injected into the mice three times every second day, and for 10 days after the first virus injection, the mice were treated with 50 mg/kg ganciclovir (GCV). 2.5 weeks after the virus treatment (5 weeks after Hep3B injection), intrapeitoneally established tumor nodules were collected, and photographed. Total mass of tumors in each mouse was then measured.

Prior to injection of adenoviruses, the mice which had been established with intraperitoneal tumor were anatomized and a carcinoma level in the peritoneal was evaluated. Injection of Hep3B cells into mice led to reproducible (>90%) and diffuse intraperitoneal tumor nodules located on the small bowel mesentery, hepatic hilum, and surface of the diaphragm. The mice with adenovirus- and GCV-treated groups were sacrificed one week after final GCV inoculation (5 weeks after Hep3B injection into mice), and their tumor growth was examined.

Statistical analysis was carried out using a statistical analysis system (SAS, SAS Institute, Cary, N.C.). Between-group differences were assessed by ANOVA. In the case of highly skewed distribution of measurements and small sample sizes, we employed nonparametric statistical tests (Kruskal-Wallis test for overall comparison and Wilcoxon rank-sum test for pair-wise comparison). All data are expressed as means±standard deviation. The significance was considered at P-values<0.05.

FIG. 12(a) is a microscopic image of the abdominal cavity of the peritoneal carcinomatosis mice, 2.5 weeks after injection of PL, PRT and CRT, and FIG. 12(b) is a microscopic image of extracted liver tissues and intraperitoneal tumor burdens.

Referring to FIG. 12, as compared to the PL/GCV-treated mice (control group), the PRT/GCV or CRT/GCV-treated mice group exhibited a significant decrease in the number and size of tumor nodules. The liver of mice treated with PRT or CRT had either tiny or no remained tumor nodules, in contrast that large tumor nodules were grown in the liver of control mice treated with PL.

FIG. 13 is a graph showing the weight of the tumor nodules 2.5 weeks after injection of Hep3B cell lines, and the weight of the tumor nodules 2.5 weeks after injection of PL, PRT and CRT (for each group, 10 mice were treated; and the average of tumor weight together with standard deviation are represented).

Referring to FIG. 13, their average masses were 0.53±0.41 g for 2.5 weeks pre-treatment group, 8.26±2.97 g for PL group, 3.52±1.99 g for PRT group and 2.36±1.39 g for CRT group. In addition, the mice group treated with adenoviruses (Ad-PEPCK.Ribo-TK, PRT) containing hTERT-targeting ribozymes exhibited significantly inhibited tumor growth (P<0.001), as compared to the mice group treated with control group adenovirus (Ad-PEPCK-LacZ, PL). These results indicate that PRT (Ad-PEPCK.Ribo-TK) exhibits inhibitory activity on tumor growth of peritoneal mice models, comparable to CRT (Ad-CMV.Ribo-TK) (P=0.1496).

### 7-2. RNA Assay

In order to confirm trans-splicing activity of ribozymes for hepatocarcinoma and liver tissues of peritoneal carcinomatosis-infected PL, PRT-administered mice, RNA assay was performed in the same manner as in Example 3.

FIG. 14(a) is RNA expression patterns of PL, PRT adenoviruses injected into normal livers (L) and several individual nodules of hepatocellular carcinomas (T), and FIG. 14(b) shows base sequence analysis results of trans-splicing molecules (TSM) generated from hepatocellular carcinomas in mice PRT-injected mice.

Referring to FIG. 14(a), as a result of the analysis to ascertain whether or not expression of suicide genes by adenoviruses in hepatocarcinomas for peritoneal carcinomatosis mouse models is due to very accurate trans-splicing reaction specific for hTERT RNA in liver cancers, PRT-injected mice expressed TK ribozymes both in tumor livers and in normal livers, while trans-splicing molecules (TSM) were generated only in tumors. This indicates that systemic delivery of adenoviruses specifically targets tumors in vivo due to trans-splicing reactions specific for hTERT. In addition, as a result of PRT administration, the difference in TSM level between individual tumors is not significant. Referring to FIG. 14(b), base sequence analysis results of trans-splicing molecules (TSM) generated in PRT-administered mice indicated that PRT correctly targeted and spliced the HSV-tk RNA onto the targeted hTERT site in the established tumor nodules.

In conclusion, adenoviruses (Ad-PEPCK.Ribo-TK) containing tumor tissue-specific promoters and the target gene specific ribozymes exhibit target tissue-specific and highly efficient anti-tumor effects through specific trans-splicing reactions. This was demonstrated by in vitro and in vivo research.

### EXAMPLE 8

### Efficient Treatment of Intrahepatic Multiple Hepatocarcinomas Through Systemically Delivery of Ad-PEPCK.Ribo-TK and GCV

Furthermore, in an attempt to establish intrahepatic multiple hepatocarcinomas mouse models and confirm anti-tumor effects of models more similar to hepatocarcinoma, the following tests were performed.

4 to 5 weeks old male BALB/cAnNCrI nude mice were etherized, the left skin was incised on the rib ends of the mice to expose the spleen, 2×10⁶ Hep3B cells in 100 μl buffer were injected under spleen capsules, pressure was applied to the site, until bleeding stopped, and the skin was sealed. After 2.5 weeks, intrahepatic multiple hepatocarcinoma mice were randomly assigned to two groups, (1) MOCK and (2) Ad-PEPCK.Ribo-TK (Each Group contains 10 mice). 2.5×10¹⁰ v.p of adenovirus was injected into the tail veins of the respective groups. For 10 days after injection of viruses, 50 mg/kg ganciclovir (GCV) was intraperitoneally injected twice daily. 2.5 weeks after treatment of viruses (a total of 5 weeks after injection of Hep3B), the peritoneum was opened, and all liver tissue was separated and then weighted.

FIG. 15 is a microscopic image of livers extracted from intrahepatic multiple hepatocarcinoma mouse models on the 10th day after Mock and PRT adenoviruses were injected.

Referring to FIG. 15, for the Mock-injected group, various sizes of tumor nodules were observed on the liver surface, but, for PRT-injected group, tumor nodules were hardly observed.

After visible photography, the liver tissues were cut to a 2 to 3 mm size and were then fixed in a 10% neutral formalin solution and paraffin blocks were manufactured by tissue-treatment. The paraffin blocks were sectioned to a thickness of 4 to 6 □m, and subjected to hematoxylin-eosin staining and to tissue photography (1:1) with an optical microscope. Then, the areas of overall liver tissues and tumor nodules were measured with a planimeter. A tumor area percentage was calculated. A tumor weight was semi-quantitatively calculated by multiplying liver weight by tumor area percentage in the liver.

FIG. 16 are optical microscopy images of intrahepatic multiple hepatocarcinoma mouse models into which Mock, PRT were administered and paraffin blocks of liver tissues were H&E stained.

Referring to FIG. 16, vivid, much and various sizes of liver tumor nodules were observed in the Mock-administered group, but, for the PRT(Ad-PEPCK.Ribo-TK)-administered group, tumors were not observed in four mice and liver tumor nodules were rarely observed in the remaining six mice.

FIG. 17 is a graph showing tumor weight of intrahepatic multiple hepatocarcinoma mouse models after administration of Mock, PRT.

Referring to FIG. 17, for the weight of tumors in the liver, the Mock-injected group was 387.29 mg in average and the PRT-injected group was 28.89 mg in average. The difference between the two groups were statistically significant (p=0.0001; Kruskal-Wallis test). That is, administration of Ad-PEPCK.Ribo-TK (PRT) efficiently induces death of tumor tissues of liver in vivo.

### INDUSTRIAL APPLICABILITY

As apparent from the foregoing, the recombinant adenoviruses of the present invention are suitable for use in anticancer agents or cancer diagnostics.

1. A recombinant adenovirus comprising:
(1) a tissue-specific promoter;
(2) a trans-splicing ribozyme acting on tumor-specific genes operably linked to the promoter; and
(3) a therapeutic or reporter gene linked to 3′ exon of the ribozyme.

2. The recombinant adenovirus according to claim 1, wherein the tissue-specific promoter is a phosphoenolpyruvate carboxykinase (PEPCK) gene promoter, an apolipoprotein E gene promoter, a serum albumin gene promoter, an alphafetoprotein (AFP) gene promoter, a carcinoembryonic antigen (CEA) gene promoter, or a prostate-specific antigen (PSA) gene promoter.

3. The recombinant adenovirus according to claim 1, further comprising:
a tissue-specific gene enhancer.

4. The recombinant adenovirus according to claim 1, wherein the tissue-specific gene enhancer is a phosphoenolpyruvate carboxykinase (PEPCK) gene promoter, an apolipoprotein E gene promoter, a serum albumin gene promoter, an alphafetoprotein (AFP) gene promoter, a carcinoembryonic antigen (CEA) gene promoter, or a prostate-specific antigen (PSA) gene promoter.

5. The recombinant adenovirus according to claim 1, wherein the tumor-specific gene is human telomerase reverse transcriptase (hTERT) mRNA, alphafetoprotein (AFP) mRNA, carcinoembryonic antigen (CEA) mRNA, prostate-specific antigen (PSA) mRNA, or cytoskeleton-associated protein 2 (CKAP2) mRNA.

6. The recombinant adenovirus according to claim 1, wherein the ribozyme is a trans-splicing Group I ribozyme specifically targeting human telomerase reverse transcriptase (hTERT) mRNA.

7. The recombinant adenovirus according to claim 1, wherein the therapeutic gene is selected from drug-sensitive genes, proapoptotic genes, cytostatic genes, cytotoxic genes, tumor suppressor genes, antigenic genes, cytokine genes, antiangiogenic genes and combinations thereof.

8. The recombinant adenovirus according to claim 1, wherein the reporter gene is a coding gene of LacZ, chloramphenicol acetyl transferase (CAT), renila luciferase, firefly luciferase, red fluorescent proteins (RFP), green fluorescent proteins (GFP), secreted placental alkaline phosphatase (SEAP) or herpes simplex virus-thymidine kinase (HSV-tk).

9. The recombinant adenovirus according to claim 1, wherein the recombinant adenovirus has a base sequence represented by Seq. No. 1 or Seq. No. 2.

10. The recombinant adenovirus according to claim 1, wherein the tissue-specific promoter is a phosphoenolpyruvate carboxykinase (PEPCK) gene promoter having a base sequence represented by Seq. No. 3.

11. The recombinant adenovirus according to claim 3, wherein the tissue-specific gene enhancer is a phosphoenolpyruvate carboxykinase (PEPCK) gene enhancer having a base sequence represented by Seq. No. 4.

12. The recombinant adenovirus according to claim 1, wherein the ribozyme is a trans-splicing Group I ribozyme specifically targeting human telomerase reverse transcriptase (hTERT) mRNA having a base sequence represented by Seq. No. 5.

13. The recombinant adenovirus according to claim 1, wherein the therapeutic gene is a herpes simplex virus-thymidine kinase (HSV-tk) gene having a base sequence represented by Seq. No. 6.

14. The recombinant adenovirus according to claim 1, wherein the reporter gene is LacZ having a base sequence represented by Seq. No. 7.

15. The recombinant adenovirus according to claim 1, wherein the recombinant adenovirus is manufactured by injection of an E1 and E3 gene-deleted adenovirus.

16. An anticancer pharmaceutical composition comprising, as an active ingredient, the recombinant adenovirus according to claim 1.

17. A composition for cancer diagnosis comprising, as an active ingredient, the recombinant adenovirus according to claim 1.

18. A cancer imaging method comprising:
S1) introducing the recombinant adenoviruses according to claim 1 into cancer cells; and
S2) detecting reporter proteins from the cancer cells.
